(19) United States
(12) Patent Application Publication
(10) Pub. No.: US 2014/0364617 A1
(43) Pub. Date: Dec. 11, 2014

(54) ANTI-VIRAL COMPOUNDS

(71) Applicant: ABBVIE INC., North Chicago, IL (US)

(72) Inventors: Allan C. Krueger, Gurnee, IL (US); Warren M. Kati, Gurnee, IL (US); Clarence J. Maring, Palatine, IL (US); Rolf Wagner, Antioch, IL (US); Charles W. Hutchins, Green Oaks, IL (US)

(21) Appl. No.: 14/368,266

(22) PCT Filed: Dec. 15, 2011
(86) PCT No.: PCT/US2011/065224

§ 371 Date: Aug. 21, 2014

Related U.S. Application Data
(60) Provisional application No. 61/425,938, filed on Dec. 22, 2010.
(60) Provisional application No. 61/423,556, filed on Dec. 15, 2010.

(51) Int. Cl. C07D 403/14; C07D 409/14

(52) U.S. Cl. CPC C07D 403/14; C07D 409/14
USPC 546/210; 548/313.1

(57) ABSTRACT

The present invention relates to anti-HCV compounds, compositions comprising the same and methods of using the same to treat HCV infection.

### FIELD

The present invention relates to anti-HCV compounds, compositions comprising the same and methods of using the same to treat HCV infection.

### BACKGROUND

Hepatitis C virus (“HCV”) is an RNA virus belonging to the Hepacivirus genus in the Flaviviridae family. The enveloped HCV virion contains a positive stranded RNA genome which encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate. Therefore, there is a need for new drugs to treat HCV infection.

### SUMMARY

In another aspect, the present invention relates to a compound of Formula (II) or pharmaceutically acceptable salts thereof:

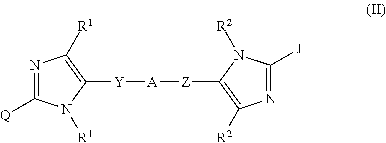

wherein:

A is (a) absent; or (b) a cyclic group independently selected from aryl, heteroaryl, heterocyclic, C₃-C₈ cycloalkyl, and C₃-C₈ cycloalkenyl, each optionally substituted; wherein A, when or if present, is preferably substituted with -L-E or -L₃-D, wherein L, E, L₃ and D are defined as below;

Y is (a) absent; or (b) an optionally substituted aliphatic group selected from the group consisting of O, S, N(R¹¹), C₁-C₈, alkyl, C₂-C₈, alkenyl, C₂-C₈, alkynyl, C₃-C₈, cycloalkyl, C₃-C₈ cycloalkenyl, and heterocyclic, each optionally substituted; or a combination thereof; wherein Y, if or when present, is optionally substituted with -L-E or -L₃-D, wherein L, E, L₃ and D are defined as below;

Z is (a) absent; or (b) an optionally substituted linear aliphatic group; optionally Z contains a group selected from O, N(R¹¹), C(O), S(O)₂, C(O)O, C(O)N(R¹¹), OC(O)O, OC(O)N(R¹¹), S(O)₂N(R¹¹), N(R¹¹)C(O)N(R¹¹), N(R¹¹)C(O)C(O)N(R¹¹), N(R¹¹)S(O)₂N(R¹¹), C(O)N(R¹¹)S(O)₂ and C(O)N(R¹¹)S(O)₂N(R¹¹); wherein Z if or when present, is optionally substituted with -L-E or -L₃-D, wherein L, E, L₃ and D are defined as below;

wherein preferably at least two of A, Y, and Z are present; and wherein at least one of A, Y and Z is substituted with -L-E or -L₃-D;

R¹¹ at each occurrence is independently hydrogen or optionally substituted C₁-C₈ alkyl;

R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —CO₂R¹¹, and —C(O)NR^{a}R^{b}; wherein at least one of R¹ and R² can be substituted with -L-E or -L₃-D wherein L, E, L₃ and D are defined as below;

R^{a} and R^{b} at each occurrence are each independently selected from the group consisting of hydrogen, optionally substituted C₁-C₈, alkyl, and optionally substituted C₂-C₈, alkenyl; or R^{a} and R^{b} can be taken together with the nitrogen atom to which they are attached to form an optionally substituted heterocyclic or optionally substituted heteroaryl group;

Q and J are each independently selected from:

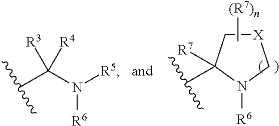

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈, cycloalkyl or optionally substituted heterocyclic;

R⁵ at each occurrence is independently hydrogen, optionally substituted C₁-C₈, alkyl, or optionally substituted C₃-C₈ cycloalkyl;

R⁶ at each occurrence is independently selected from the group consisting of —C(O)—R², —C(O)—C(O)—R¹², —S(O)₂—R¹², and —C(S)—R¹²;

R¹² at each occurrence is independently selected from the group consisting of: optionally substituted C₁-C₈ alkyl, —O—R¹¹, —NR^{a}R^{b}, —R¹³, and —NR^{c}R^{d};

R¹³ at each occurrence is independently selected from the group consisting of hydrogen, C₁-C₈ alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and

R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R¹³)₈;

m is 0, 1, or 2;

n is 1, 2, 3, or 4;

X at each occurrence is independently selected from O, S, S(O), SO₂, and C(R⁷)₂ provided that when m is 0, X is C(R⁷)₂; and

R⁷ at each occurrence is independently selected from the group consisting of hydrogen, halogen, —C₁-C₄ alkyl, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; optionally substituted cyclopropyl; or alternatively two geminal R⁷ groups can be taken together with the carbon atom to which they are attached to form a spiro, optionally substituted C₃-C₈ cycloalkyl or optionally substituted heterocyclic ring,

-L-E are as follows:

E is (i) C₃-C₁₄ carbocycle or 3- to 14-membered heterocycle, and is optionally substituted with one or more R_{A}; or (ii) E is -L_{S}-R_{E};

L is -L_{S}-, -L_{S}-O-L_{S}′, -L_{S}-C(O)-L_{S}′-, -L_{S}-S(O)₂-L_{S}′, -L_{S}-S(O)-L_{S}′-, -L_{S}-OS(O)₂-L_{S}′-, -L_{S}-S(O)₂O-L_{S}′, -L_{S}-OS(O)-L_{S}′-, -L_{S}-S(O)O-L_{S}′-, -L_{S}-C(O)O-L_{S}′-, -L_{S}-OC(O)-L_{S}′-, -L_{S}-OC(O)O-L_{S}′-, -L_{S}-C(O)N(R_{B})-L_{S}′-, -L_{S}-N(R_{B})C(O)-L_{S}′-, -L_{S}-C(O)N(R_{B})O-L_{S}′-, -L_{S}-N(R_{B})C(O)O-L_{S}′-, -L_{S}-OC(O)N(R_{B})-L_{S}′-, -L_{S}-C(O)N(R_{B})N(R_{B})-L_{S}′-, -L_{S}-S-L_{S}′-, -L_{S}-C(S)-L_{S}′-, -L_{S}-C(S)O-L_{S}′-, -L_{S}-OC(S)-L_{S}′-, -L_{S}-C(S)N(R_{B})-L_{S}′-, -L_{S}-N(R_{B})-L_{S}′-, -L_{S}-N(R_{B})C(S)-L_{S}′-, -L_{S}-N(R_{B})S(O)-L_{S}′-, -L_{S}-N(R_{B})S(O)₂-L_{S}′-, -L_{S}-S(O)₂N(R₈)-L_{S}′-, -L_{S}-S(O)N(R_{B})-L_{S}′-, -L_{S}-C(S)N(R_{B})O-L_{S}′-, -L_{S}-C(O)N(R)C(O)-L_{S}′-, -L_{S}-N(R_{B})C(O)N(R_{B}′)-L_{S}′-, -L_{S}-N(R_{B})SO₂N(R_{B}′)-L_{S}′-, -L_{S}-N(R_{B})S(O)N(R_{B}′)-L_{S}′-, or -L_{S}-C(S)N(R_{B})N(R_{B}′)-L_{S}′-;

L_{S} and L_{S}′ are each independently selected at each occurrence from bond; or C₁-C₆ alkylene, C₂-C₆ alkenylene or C₂-C₆ alkynylene, each of which is independently optionally substituted at each occurrence with one or more R_{L};

R_{A} is independently selected at each occurrence from halogen, oxo, thioxo, hydroxy, mercapto, nitro, cyano, amino, carboxy, formyl, phosphonoxy, or phosphono; or -L_{S}-R_{E};

R_{B} and R_{B′} are each independently selected at each occurrence from hydrogen; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆ carbocycle or 3- to 6-membered heterocycle; or C₃-C₆ carbocycle or 3- to 6-membered heterocycle; wherein each C₃-C₆ carbocycle or 3- to 6-membered heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;

R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′) or —C(O)N(R_{S})C(O)—R_{S}′; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;

R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆ carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;

R_{S}, R_{S}′ and R_{S}″ are each independently selected at each occurrence from hydrogen; C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;

-L₃-D are as follows:

L₃ is bond or -L_{S}-K-L_{S}′-, wherein K is selected from bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, or —N(R_{B})S(O)N(R_{B}′)—;

D is C₃-C₁₂ carbocycle or 3- to 12-membered heterocycle, and is optionally substituted with one or more R_{A}; or D is C₃-C₁₂ carbocycle or 3- to 12-membered heterocycle which is substituted with J and optionally substituted with one or more R_{A}, where J is C₃-C₁₂ carbocycle or 3- to 12-membered heterocycle and is optionally substituted with one or more R_{A}, or J is —SF₅; or D is hydrogen or R_{A};

R_{A} is independently selected at each occurrence from halogen, nitro, oxo, phosphonoxy, phosphono, thioxo, cyano, or -L_{S}-R_{E}, wherein two adjacent R_{A}, taken together with the atoms to which they are attached and any atoms between the atoms to which they are attached, can optionally form carbocycle or heterocycle;

R_{B} and R_{B}′ are each independently selected at each occurrence from hydrogen; or C₁-C₆alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;

R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′-N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, or —C(O)N(R_{S})C(O)—R_{S}′; or C₁-C₆alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆ carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆alkenyl, C₂-C₆ alkynyl, C₁-C₆ haloalkyl, C₂-C₆ haloalkenyl, C₂-C₆ haloalkynyl, C(O)OR_{S}, or —N(R_{S}R_{S}′);

R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆ carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl; wherein two adjacent R_{L}, taken together with the atoms to which they are attached and any atoms between the atoms to which they are attached, can optionally form carbocycle or heterocycle;

L_{S} and L_{S}′ are each independently selected at each occurrence from bond; or C₁-C₆alkylene, C₂-C₆alkenylene or C₂-C₆alkynylene, each of which is independently optionally substituted at each occurrence with one or more R_{L}; and

R_{S}, R_{S}′ and R_{S}″ are each independently selected at each occurrence from hydrogen; C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —O—C₁-C₆ alkyl, —O—C₁-C₆ alkylene-O—C₁-C₆ alkyl, or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl.

In still yet another aspect, the present invention relates to a compound of Formula (V) or pharmaceutically acceptable salts thereof:

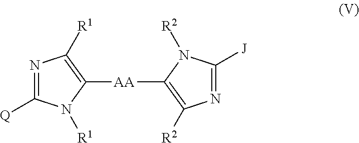

wherein:
AA is an (a) aromatic group selected from the group consisting of:
i. a bicyclic aryl or bicyclic heteroaryl, wherein the rings of said bicyclic ring system are fused or covalently attached, wherein each imidazole moiety is attached to a different ring of said bicyclic ring system and wherein at least one ring of said bicyclic is a 5- or 7-membered ring;
ii. a tricyclic aryl or tricyclic heteroaryl, wherein the rings of said tricyclic ring system are fused, covalently attached or a combination thereof, and wherein each imidazole moiety is attached to a different ring of said tricyclic; and

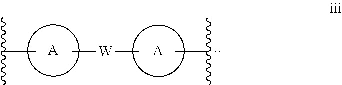

each A is independently a 5-7 membered aryl or heteroaryl, wherein said aryl or heteroaryl are optionally substituted; or

wherein AA is substituted with -L-E or preferably -L₃-D, wherein L, E, L₃ and D are defined as hereinabove and below in connection with the compounds of Formula II;

W is an aliphatic group containing zero to eight carbons;

R¹¹ at each occurrence is independently hydrogen or optionally substituted C₁-C₈ alkyl;

R¹ and R² at each occurrence are each independently selected from the group consisting of: halogen, cyano, optionally substituted C₁-C₄ alkyl, —O—R¹¹, —NR^{a}R^{b}, —C(O)R¹¹, —CO₂R¹¹, and —C(O)NR^{a}R^{b}; wherein at least one of R¹ and R² are substituted with -L-E or -L₃-D as defined above in connection with the compounds of Formula II;

R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;

Q and J are each independently selected from:

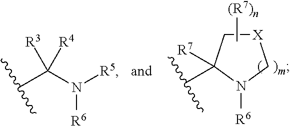

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈ cycloalkyl or optionally substituted heterocyclic;

R⁵ at each occurrence is independently hydrogen, optionally substituted C₁-C₈ alkyl, or optionally substituted C₃-C₈ cycloalkyl;

R⁶ at each occurrence is independently, selected from the group consisting of —C(O)—R¹², —C(O)—C(O)—R¹², —S(O)₂—R¹², and —C(S)—R¹²;

R¹² at each occurrence is independently selected from the group consisting of: —O—R¹¹, —NR^{a}—R^{b}, —R¹³, —NR^{c}R^{d};

R¹³ at each occurrence is independently selected from the group consisting of hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and

R^{c} and R^{d} at each occurrence are each independently selected from the group consisting of hydrogen, —R¹³, —C(O)—R¹³, —C(O)—OR³, —S(O)₂—R¹³, —C(O)N(R¹³)₂, and —S(O)₂N(R¹³)₂;

m is 0, 1, or 2;

n is 1, 2, 3, or 4;

X at each occurrence is independently selected from O, S, S(O), SO₂, and C(R⁷)₂; provided that when m is 0, X is C(R⁷)₂; and

R⁷ at each occurrence is independently selected from the group consisting of hydrogen, halogen, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; or alternatively two geminal R⁷ groups can be taken together with the carbon atom to which they are attached to form a spiro, optionally substituted C₃-C₈ cycloalkyl or optionally substituted heterocyclic ring.

In still yet another aspect, the present invention relates to a compound of Formula (VI) or pharmaceutically acceptable salts thereof:

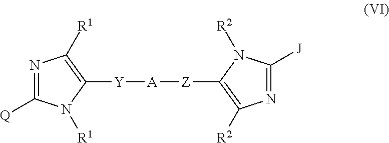

A is (a) absent; or (b) a cyclic group independently selected from aryl, heteroaryl, heterocyclic, C₃-C₈ cycloalkyl, and C₃-C₈ cycloalkenyl, each optionally substituted; wherein A, if or when present, is preferably substituted with -L-E or -L₃-D, wherein L, E, L₃ and D are defined above in connection with the compounds of Formula II;

Y is (a) absent; or (b) an optionally substituted aliphatic group selected from the group consisting of O, S, N(R¹¹), C₁-C₈ alkyl, C₂-C₈ alkenyl, C₂-C₈ alkynyl, C₃-C₈ cycloalkyl, C₃-C₈ cycloalkenyl, and heterocyclic, each optionally substituted; or a combination thereof; wherein Y, if or when present, is optionally substituted with -L-E or -L₃-D, wherein L, E, L₃ and D are defined above above in connection with the compounds of Formula II;

Z is (a) absent; or (b) an optionally substituted linear aliphatic group; optionally Z contains a group selected from O, N(R¹¹), C(O), S(O)₂, C(O)O, C(O)N(R¹¹), OC(O)O, OC(O)N(R¹¹), S(O)₂N(R¹¹), N(R¹¹)C(O)N(R¹¹), N(R¹¹)C(O)C(O)N(R¹¹), N(R¹¹)S(O)₂N(R¹¹), C(O)N(R¹¹)S(O)₂ and C(O)N(R¹¹)S(O)₂N(R¹¹); wherein Z, if or when present, is optionally substituted with -L-E or -L₃-D, wherein L, E, L₃ and D are defined above in connection with the compounds of Formula II;

wherein preferably at least two of A, Y, and Z are present; and wherein at least one of A, Y and Z is substituted with -L-E or -L₃-D;

R¹¹ at each occurrence is independently hydrogen or optionally substituted C₁-C₈ alkyl;

R¹ and R² at each occurrence are (a) each independently selected from the group consisting of hydrogen, halogen, cyano, optionally substituted C₁-C₄ alkyl, —O—R¹¹, —NR^{a}R^{b}, —C(O)R¹¹, —C(O)₂R¹¹, and —C(O)NR^{a}R^{b}; wherein at least one of R¹ and R² can be substituted with -L-E or -L₃-D, wherein L, E, L₃ and D are defined above in connection with the compounds of Formula II;

R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;

Q and J are each independently selected from:

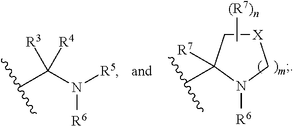

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈ cycloalkyl or optionally substituted heterocyclic;

R⁵ at each occurrence is independently hydrogen, optionally substituted C₁-C₈ alkyl, or optionally substituted C₃-C₈ cycloalkyl;

R⁶ at each occurrence is independently selected from the group consisting of —C(O)—R¹², —C(O)—C(O)—R¹², —S(O)₂—R¹², and —C(S)—R¹²;

R¹² at each occurrence is independently selected from the group consisting of: —O—R¹¹, —NR^{a}R^{b}, —R¹³, and —NR^{c}R^{d};

R¹³ at each occurrence is independently selected from the group consisting of hydrogen, C₁-C₈ alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and

R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R¹³)₂;

m is 0, 1, or 2;

n is 1, 2, 3, or 4;

X at each occurrence is independently selected from O, S, S(O), SO₂, and C(R⁷)₂; provided that when m is 0, X is C(R⁷)₂; and

R⁷ at each occurrence is independently selected from the group consisting of hydrogen, halogen, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; or alternatively two geminal R⁷ groups can be taken together with the carbon atom to which they are attached to form a spiro, optionally substituted C₃-C₈ cycloalkyl or optionally substituted heterocyclic ring.

In still yet another aspect, the present invention relates to a compound of Formula (VII) or pharmaceutically acceptable salts thereof:

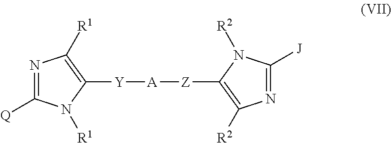

wherein:

A is (a) absent or (b) is selected from the group consisting of an optionally substituted aliphatic group, an optionally substituted aryl, an optionally substituted heteroaryl, and a combination thereof; wherein A, when present, is preferably substituted with -L-E or -L₃-D as defined above in connection with the compounds of Formula II;

Y and Z at each occurrence are each independently selected from —C(O)—, —C(O)O—, —O(O)C—, —C(O)N(R^{11b})—, —N(R^{11b})C(O)—, —S(O₂)N(R^{11b})—, —N(R^{11b})S(O₂), —N(R^{11b})C(O)O—, —OC(O)N(R^{11b})—, and —N(R^{11b})C(O)N(R^{11c})—; wherein at least one of A, Y or Z is substituted with -L-E or -L₃-D as defined above in connection with the compounds of Formula II;

R^{11b} and R^{11c} at each occurrence is independently hydrogen or optionally substituted C₁-C₈ alkyl;

R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —CO₂R¹¹, and —C(O)NR^{a}R^{b}; wherein at least one of R¹ and R² can be substituted with -L-E or -L₃-D as defined above in connection with the compounds of Formula II;

R¹¹ at each occurrence is independently hydrogen or optionally substituted C₁-C₈ alkyl;

R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;

Q and J are each independently selected from:

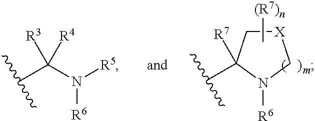

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈ cycloalkyl or optionally substituted heterocyclic;

R⁵ at each occurrence is independently hydrogen, optionally substituted C₁-C₈ alkyl, or optionally substituted C₃-C₈ cycloalkyl;

R⁶ is selected from the group consisting of —C(O)—R¹², —C(O)—C(O)—R², —S(O)₂—R¹², and —C(S)—R¹²;

R¹² at each occurrence is independently selected from the group consisting of: —O—R¹¹, NR^{a}R^{b}, —R¹³, and —NR^{c}R^{d};

R¹¹ at each occurrence is independently selected from the group consisting of: hydrogen, C₁-C₈ alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and

R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R¹³)₂;

m is 0, 1, or 2;

n is 1, 2, 3, or 4;

X is selected from O, S, S(O), SO₂, and C(R⁷)₂; provided that when m is 0, X is C(R⁷)₂; and

R⁷ at each occurrence is independently selected from the group consisting of: hydrogen, halogen, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; or alternatively two geminal R⁷ groups can be taken together with the carbon atom to which they are attached to form a spiro, optionally substituted C₃-C₈ cycloalkyl or optionally substituted heterocyclic ring.

In yet another aspect, the present invention relates to a pharmaceutical composition comprising (a) one or more of any of the compounds of Formula (II), Formula (V), Formula (VI) or Formula (VII) or any salts, solvates or prodrugs thereof; and (b) at least one pharmaceutically acceptable carrier or at least one pharmaceutically acceptable excipient. Examples of suitable pharmaceutically acceptable carriers or excipients that can be used in said pharmaceutical compositions include, but are not limited to, sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, phosphate buffer solutions, lubricants, coloring agents, releasing agents, coating agents, sweetening, flavoring or perfuming agents, preservatives, or antioxidants.

In addition to containing any one or more compounds of Formula (II), Formula (V), Formula (VI) or Formula (VII) or any salts, solvates or prodrugs thereof, the pharmaceutical compositions of the present invention can also further contain one or more of the following: (a) one or more anti-HCV agents, such as an HCV polymerase inhibitor, HCV protease inhibitor, HCV helicase inhibitor, CD81 inhibitor, cyclophilin inhibitors, IRES inhibitors, or NS5A inhibitors; (b) one or more antiviral agents such as anti-HBV agents, anti-HIV agents, anti-hepatitis agents, anti-hepatitis D, anti-hepatitis E or anti-hepatitis G agents; (c) anti-bacterial agents; (d) anti-fungal agents; (e) immunomodulators, (f) anti-cancer or chemotherapeutic agents; (g) anti-inflammatory agents; (h) antisense RNA; (i) antibodies; (j) agents for treating cirrhosis or inflammation of the liver; or (k) any combinations of (a)-(k).

The present invention also relates to a method of treating HCV infection. The method involves administering to a patient in need of treatment, a therapeutically effective amount of the above-described pharmaceutical composition of the present invention to treat the HCV infection in said patient.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### DETAILED DESCRIPTION

In an aspect, the present invention relates to compounds having the structure of below Formula (II) or pharmaceutically acceptable salts thereof:

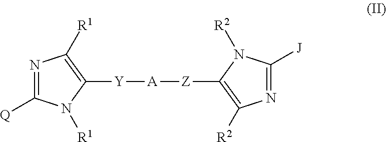

wherein:

A is (a) absent; or (b) a cyclic group independently selected from aryl, heteroaryl, heterocyclic, C₃-C₈ cycloalkyl, and C₃-C₈ cycloalkenyl, each optionally substituted; wherein A, if or when present, preferably is substituted with -L-E or -L₃-D as defined hereinabove and below;

Y is (a) absent; or (b) an optionally substituted aliphatic group selected from the group consisting of O, S, N(R¹¹), C₁-C₈, alkyl, C₂-C₈, alkenyl, C₂-C₈, alkynyl, C₃-C₈, cycloalkyl, C₃-C₈ cycloalkenyl, and heterocyclic, each optionally substituted; or a combination thereof; wherein Y, if or when present, is optionally substituted with -L-E or -L₃-D as defined hereinabove and below;

Z is (a) absent; or (b) an optionally substituted linear aliphatic group; optionally Z contains a group selected from O, N(R¹¹), C(O), S(O)₂, C(O)O, C(O)N(R¹¹), OC(O)O, OC(O)N(R¹¹), S(O)₂N(R¹¹), N(R¹¹)C(O)N(R¹¹), N(R¹¹)C(O)C(O)N(R¹¹), N(R¹¹)S(O)₂N(R¹¹), C(O)N(R¹¹)S(O)₂ and C(O)N(R¹¹)S(O)₂N(R¹¹); wherein Z, if present, is optionally substituted as defined hereinabove and below;

wherein preferably at least two of A, Y, and Z are optionally present; and wherein at least one of A, Y and Z is substituted with -L-E or -L₃-D;

R¹¹ at each occurrence is independently hydrogen or optionally substituted C₁-C₈ alkyl;

R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —CO₂R¹¹, and —C(O)NR^{a}R^{b}; preferably hydrogen, halogen and optionally substituted C₁-C₄ alkyl; wherein at least one of R¹ and R² is independently substituted with -L-E or -L₃-D as defined below;

R^{a} and R^{b} at each occurrence are each independently selected from the group consisting of hydrogen, optionally substituted C₁-C₈, alkyl, and optionally substituted C₂-C₈, alkenyl; or R^{a} and R^{b} can be taken together with the nitrogen atom to which they are attached to form an optionally substituted heterocyclic or optionally substituted heteroaryl group;

Q and J are each independently selected from:

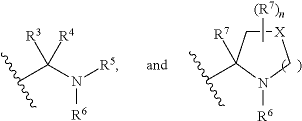

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; preferably hydrogen or optionally substituted C₁-C₄ alkyl; or alternatively, R³ and R⁴ can be taken together with the carbon atom to which they are attached to form optionally substituted C₃-C₈, cycloalkyl or optionally substituted heterocyclic;

R⁵ at each occurrence is independently hydrogen, optionally substituted C₁-C₈, alkyl, or optionally substituted C₃-C₈ cycloalkyl; preferably hydrogen or optionally substituted C₁-C₄ alkyl;

R⁶ at each occurrence is independently selected from the group consisting of —C(O)—R¹², —C(O)—C(O)—R¹², —S(O)₂—R¹², and —C(S)—R¹², preferably —C(O)—R¹², more preferably an optionally substituted amino acid acyl;

R¹² at each occurrence is independently selected from the group consisting of: optionally substituted C₁-C₈ alkyl, —O—R¹¹, —NR^{a}R^{b}, —R³, and —NR^{c}R^{d}, preferably optionally substituted C₁-C₈ alkyl and —O—R¹¹;

R¹³ at each occurrence is independently selected from the group consisting of hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; preferably optionally substituted C₁-C₈ alkyl; more preferably C₁-C₈ alkyl optionally substituted with amino, hydroxy, optionally substituted phenyl, protected amino, or O(C₁-C₄ alkyl); and

R^{c} and R^{d} at each occurrence are each independently selected from the group consisting of hydrogen, —R³, —C(O)—R¹³, —C(O)—OR¹³, —S(O)₂—R¹³, —C(O)N(R¹³)₂, and —S(O)₂N(R¹³)₈;

m is 0, 1, or 2, preferably 1;

n is 1, 2, 3, or 4, preferably 1 or 2;

X at each occurrence is independently selected from O, S, S(O), SO₂, and C(R⁷)₂, preferably CH₂or CHR⁷; provided that when m is 0, X is C(R⁷)₂; and

R⁷ at each occurrence is independently selected from the group consisting of hydrogen, halogen, —C₁-C₄ alkyl, cyano, —O—R¹¹, —NR^{a}R^{b}, optionally substituted aryl, optionally substituted heteroaryl, and optionally substituted —C₁-C₄ alkyl; preferably hydrogen, methyl or halogen; or two vicinal R⁷ groups can be taken together with the two adjacent atoms to which they are attached to form a fused, optionally substituted C₃-C₈ cycloalkyl or optionally substituted heterocyclic ring; preferably a fused, optionally substituted cyclopropyl; or alternatively two geminal R⁷ groups can be taken together with the carbon atom to which they are attached to form a spiro, optionally substituted C₃-C₈ cycloalkyl or optionally substituted heterocyclic ring; preferably a spiro, optionally substituted cyclopropyl.

With respect to -L-E as used herein:

E is (i) C₃-C₁₄ carbocycle or 3- to 14-membered heterocycle, and is optionally substituted with one or more R_{A}; or (ii) E is -L_{S}-R_{E};

L is -L_{S}-, -L_{S}-O-L_{S}′, -L_{S}-C(O)-L_{S}′-, -L_{S}-S(O)₂-L_{S}′, -L_{S}-S(O)-L_{S}′-, -L_{S}-OS(O)₂-L_{S}′, -L_{S}-S(O)₂O-L_{S}′-, -L_{S}-OS(O)-L_{S}′-, -L_{S}-S(O)O-L_{S}′-, -L_{S}-C(O)O-L_{S}′-, -L_{S}-OC(O)-L_{S}′-, -L_{S}-OC(O)O-L_{S}′-, -L_{S}-C(O)N(R_{B})-L_{S}′-, -L_{S}-N(R_{B})C(O)-L_{S}′-, -L_{S}-C(O)N(R_{B})O-L_{S}′-, -L_{S}-N(R_{B})C(O)O-L_{S}′-, -L_{S}-OC(O)N(R_{B})-L_{S}′-, -L_{S}-C(O)N(R_{B})N(R_{B})-L_{S}′-, -L_{S}-S-L_{S}′-, -L_{S}-C(S)-L_{S}′-, -L_{S}-C(S)O-L_{S}′-, -L_{S}-OC(S)-L_{S}′-, -L_{S}-C(S)N(R_{B})-L_{S}′-, -L_{S}-N(R_{B})-L_{S}′-, -L_{S}-N(R_{B})C(S)-L_{S}′-, -L_{S}-N(R_{B})S(O)-L_{S}′-, -L_{S}-N(R_{B})S(O)₂-L_{S}′-, -L_{S}-S(O)₂N(R_{B})-L_{S}′-, -L_{S}-S(O)N(R_{B})-L_{S}′-, -L_{S}-C(S)N(R_{B})O-L_{S}′-, -L_{S}-C(O)N(R_{B})C(O)-L_{S}′-, -L_{S}-N(R_{B})C(O)N(R_{B}′)-L_{S}′-, -L_{S}-N(R_{B})SO₂N(R_{B}′)-L_{S}′-, -L_{S}-N(R_{B})S(O)N(R_{B}′)-L_{S}′-, or -L_{S}-C(S)N(R_{B})N(R_{B})-L_{S}′-;

L_{S} and L_{S}′ are each independently selected at each occurrence from bond; or C₁-C₆ alkylene, C₂-C₆ alkenylene or C₂-C₆ alkynylene, each of which is independently optionally substituted at each occurrence with one or more R_{L};

R_{A} is independently selected at each occurrence from halogen, oxo, thioxo, hydroxy, mercapto, nitro, cyano, amino, carboxy, formyl, phosphonoxy, or phosphono; or -L_{S}-R_{E};

R_{B} and R_{B′} are each independently selected at each occurrence from hydrogen; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆ carbocycle or 3- to 6-membered heterocycle; or C₃-C₆ carbocycle or 3- to 6-membered heterocycle; wherein each C₃-C₆ carbocycle or 3- to 6-membered heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆ haloalkyl, C₂-C₆haloalkenyl or C₂-C₆ haloalkynyl;

R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′-N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′) or —C(O)N(R_{S})C(O)—R_{S}′; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆ carbocycle or 3- to 6-membered heterocycle, each of which is independently optionally substituted at each occurrence with one or more substituents selected, from halogen, hydroxy, mercapto, amino, carboxy, nitro, oxo, phosphonoxy, phosphono, thioxo, formyl, cyano, C₁-C₆alkyl, C₂-C₆ alkenyl, C₂-C₆ alkynyl, C₁-C₆ haloalkyl, C₂-C₆ haloalkenyl or C₂-C₆ haloalkynyl;

R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆ carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;

R_{S}, R_{S}′ and R_{S}″ are each independently selected at each occurrence from hydrogen; C₁-C₆alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆ alkenyl, C₂-C₆ alkynyl, C₁-C₆ haloalkyl, C₂-C₆ haloalkenyl or C₂-C₆haloalkynyl;

For -L₃-D:

L₃ is bond or -L_{S}-K-L_{S}′-, wherein K is selected from bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, or —N(R_{B})S(O)N(R_{B}′)—; preferably, L₃ is bond, C₁-C₆alkylene, C₂-C₆alkenylene or C₂-C₆alkynylene; more preferably, L₃ is bond;

D is C₃-C₁₂ carbocycle or 3- to 12-membered heterocycle, and is optionally substituted with one or more R_{A}; or D is C₃-C₁₂ carbocycle or 3- to 12-membered heterocycle which is substituted with J and optionally substituted with one or more R_{A}, where J is C₃-C₂ carbocycle or 3- to 12-membered heterocycle and is optionally substituted with one or more R_{A}, or J is —SF₅; or D is hydrogen or R_{A};

R_{A} is independently selected at each occurrence from halogen, nitro, oxo, phosphonoxy, phosphono, thioxo, cyano, or -L_{S}-R_{E}, wherein two adjacent R_{A}, taken together with the atoms to which they are attached and any atoms between the atoms to which they are attached, can optionally form carbocycle or heterocycle;

R_{B} and R_{B}′ are each independently selected at each occurrence from hydrogen; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;

R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, or —C(O)N(R_{S})C(O)—R_{S}′; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆ carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆ alkynyl, C₁-C₆ haloalkyl, C₂-C₆ haloalkenyl, C₂-C₆ haloalkynyl, C(O)OR_{S}, or —N(R_{S}R_{S}′);

R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆ carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆ alkenyl, C₂-C₆ alkynyl, C₁-C₆ haloalkyl, C₂-C₆ haloalkenyl or C₂-C₆ haloalkynyl; wherein two adjacent R_{L}, taken together with the atoms to which they are attached and any atoms between the atoms to which they are attached, can optionally form carbocycle or heterocycle;

L_{S} and L_{S}′ are each independently selected at each occurrence from bond; or C₁-C₆alkylene, C₂-C₆alkenylene or C₂-C₆alkynylene, each of which is independently optionally substituted at each occurrence with one or more R_{L}; and

R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆alkylene-O—C₁-C₆alkyl, or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl.

Preferably, -L-E comprises C₅-C₆ carbocycle, 5- to 6-membered heterocycle, or 6- to 12-membered bicycle, each of which is optionally substituted with one or more R_{A} as defined above. Also preferably, the moiety comprises C₁-C₆ alkyl, C₂-C₆ alkenyl or C₂-C₆ alkynyl, each of which is optionally substituted with one or more R_{L} as defined above. More preferably, the moiety comprises C₅-C₆carbocycle, 5- to 6-membered heterocycle, or 6- to 12-membered bicycles, each of which is optionally substituted with one or more substituents selected from halogen, hydroxy, mercapto, amino, carboxy, nitro, oxo, phosphonoxy, phosphono, thioxo, cyano, C₁-C₆alkyl, C₂-C₆ alkenyl or C₂-C₆ alkynyl, wherein each of said C₁-C₆ alkyl, C₂-C₆ alkenyl or C₂-C₆ alkynyl can be further independently optionally substituted at each occurrence with one or more substituents selected from halogen, hydroxy, mercapto, amino, carboxy, nitro, oxo, phosphonoxy, phosphono, thioxo, formyl, cyano, C₃-C₆ carbocycle or 3- to 6-membered heterocycle. Highly preferably, the moiety comprises C₅-C₆ carbocycle, 5- to 6-membered heterocycle, or 6- to 12-membered bicycles, each of which is optionally substituted with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆haloalkenyl or C₂-C₆ haloalkynyl.

In one example, -L-E comprises phenyl optionally substituted with one or more substituents selected from is halogen, hydroxy, mercapto, amino, carboxy, C₁-C₆ alkyl, C₂-C₆ alkenyl or C₂-C₆ alkynyl, wherein each of said C₁-C₆ alkyl, C₂-C₆ alkenyl or C₂-C₆ alkynyl is independently optionally substituted at each occurrence with one or more substituents selected from halogen, hydroxy, mercapto, amino or carboxy. In another example, the moiety comprises C₁-C₆ alkyl, C₂-C₆ alkenyl or C₂-C₆ alkynyl, each of which is optionally substituted with one or more substituents selected from halogen, hydroxy, mercapto, amino, carboxy, nitro, oxo, phosphonoxy, phosphono, thioxo, formyl or cyano.

In the above Formula II, D in -L₃-D preferably is selected from C₅-C₆ carbocycle, 5- to 6-membered heterocycle, or 6- to 12-membered bicycles, and is optionally substituted with one or more R_{A}. D can also be preferably selected from C₁-C₆ alkyl, C₂-C₆ alkenyl or C₂-C₆ alkynyl, and is optionally substituted with one or more substituents selected from R_{L}. More preferably, D is C₅-C₆carbocycle (e.g., phenyl), 5- to 6-membered heterocycle (e.g., pyridinyl, pyrimidinyl, thiazolyl), or 6- to 12-membered bicycles (e.g., indanyl, 4,5,6,7-tetrahydrobenzo[d]thiazolyl, benzo[d]thiazolyl, indazolyl, benzo[d][1,3]dioxol-5-yl), and is substituted with one or more R_{M}, where R_{M} is halogen, nitro, oxo, phosphonoxy, phosphono, thioxo, cyano, or -L_{S}-R_{E}. Also preferably, D is phenyl, and is optionally substituted with one or more R_{A}. More preferably, D is phenyl, and is substituted with one or more R_{M}, wherein R_{M} is as defined above. Highly preferably, D is

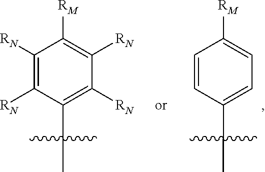

wherein R_{M} is as defined above, and each R_{N} is independently selected from R_{D} and preferably is hydrogen. One or more R_{N} can also preferably be halo such as F.

D is also preferably pyridinyl, pyrimidinyl, or thiazolyl, optionally substituted with one or more R_{A}. More preferably D is pyridinyl, pyrimidinyl, or thiazolyl, and is substituted with one or more R_{M}. Highly preferably, D is

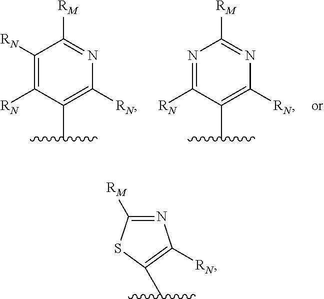

wherein R_{M} is as defined above, and each R_{N} is independently selected from R_{D} and preferably is hydrogen. One or more R_{N} can also preferably be halo such as F. D is also preferably indanyl, 4,5,6,7-tetrahydrobenzo[d]thiazolyl, benzo[d]thiazolyl, or indazolyl, and is optionally substituted with one or more R_{A}. More preferably D is indanyl, 4,5,6,7-tetrahydrobenzo[d]thiazolyl, benzo[d]thiazolyl, indazolyl, or benzo[d][1,3]dioxol-5-yl,

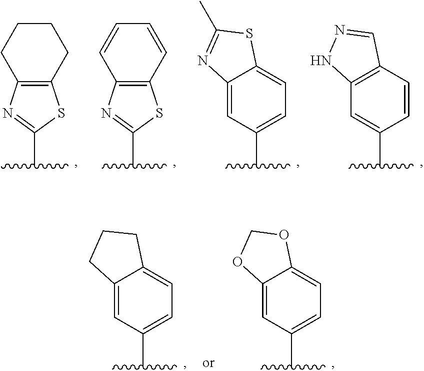

and is optionally substituted with one or more R_{M}.

Preferably, R_{M} is halogen, hydroxy, mercapto, amino, carboxy, nitro, oxo, phosphonoxy, phosphono, thioxo, cyano; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆alkynyl, C₁-C₆ haloalkyl, C₂-C₆ haloalkenyl or C₂-C₆ haloalkynyl. More preferably, R_{M} is halogen, hydroxy, mercapto, amino, carboxy; or C₁-C₆alkyl, C₂-C₆alkenyl or C₂-C₆ alkynyl, each of which is independently optionally substituted at each occurrence with one or more substituents selected from halogen, hydroxy, mercapto, amino or carboxy. Highly preferably, R_{M} is C₁-C₆ alkyl which is optionally substituted with one or more substituents selected from halogen, hydroxy, mercapto, amino or carboxy.

Also preferably, R_{M} is halogen, hydroxy, mercapto, amino, carboxy, nitro, oxo, phosphonoxy, phosphono, thioxo, or cyano; or R_{M} is -L_{S}-R_{E}, wherein L_{S} is a bond or C₁-C₆alkylene, and R_{E} is —N(R_{S}R_{S}′), —O—R_{S}, —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —SO₂R_{S}, —SR₅, or —P(O)(OR_{S})₂, wherein R_{S} and R_{S}′ can be, for example, each independently selected at each occurrence from (1) hydrogen or (2) C₁-C₆ alkyl optionally substituted at each occurrence with one or more halogen, hydroxy, —O—C₁-C₆alkyl or 3- to 6-membered heterocycle; or R_{M} is C₁-C₆ alkyl, C₂-C₆ alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or R_{M} is C₃-C₆ carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆ haloalkenyl, C₂-C₆ haloalkynyl, —C(O)OR_{S}, or —N(R_{S}R_{S}′). More preferably, R_{M} is halogen (e.g., fluoro, chloro, bromo, iodo), hydroxy, mercapto, amino, carboxy, or C₁-C₆alkyl (e.g., methyl, isopropyl, tert-butyl), C₂-C₆alkenyl or C₂-C₆alkynyl, each of which is independently optionally substituted at each occurrence with one or more substituents selected from halogen, hydroxy, mercapto, amino, cyano, or carboxy. For example, R_{M} is CF₃, —C(CF₃)₂—OH, —C(CH₃)₂—CN, —C(CH₃)₂—CH₂OH, or —C(CH₃)₂—CH₂NH₂. Also preferably R_{M} is -L_{S}-R_{E} where L_{S} is a bond and R_{E} is —N(R_{S}R_{S′}), —O—R_{S}, —N(R_{S})C(O)OR_{S}′, —N(R_{S})SO₂R_{S}′, —SO₂R_{S}, or —SR₅. For example where L_{S} is a bond, R_{E} is —N(C₁-C₆alkyl)₂ (e.g., —NMe₂); —N(C₁-C₆ alkylene-O—C₁-C₆ alkyl)₂ (e.g. —N(CH₂CH₂OMe)₂); —N(C₁-C₆ alkyl)(C₁-C₆ alkylene-O—C₁-C₆ alkyl) (e.g. —N(CH₃)(CH₂CH₂OMe)); —O—C₁-C₆alkyl (e.g., —O-Me, —O-Et, —O— isopropyl, —O-tert-butyl, —O-n-hexyl); —O—C₁-C₆ haloalky 1 (e.g., —OCF₃, —OCH₂CF₃); —O—C₁-C₆alkylene-piperidine (e.g., —O—CH₂CH₂-1-piperidyl); —N(C₁-C₆alkyl)C(O)O—C₁-C₆ alkyl (e.g., —N(CH₃)C(O)O—CH₂CH(CH₃)₂), —N(C₁-C₆ alkyl)SO₂C₁-C₆ alkyl (e.g., —N(CH₃)SO₂CH₃); —SO₂C₁-C₆ alkyl (e.g., —SO₂Me); —SO₂C₁-C₆ haloalkyl (e.g., —SO₂CF₃); or —S—C₁-C₆ haloalkyl (e.g., SCF₃). Also preferably R_{M} is -L_{S}-R_{E} where L_{S} is C₁-C₆ alkylene (e.g., —CH₂—, —C(CH₃)₂—, —C(CH₃)₂—CH₂—) and R_{E} is —O—R_{S}, —C(O)OR_{S}, —N(R_{S})C(O)OR_{S}′, or —P(O)(OR_{S})₂. For example R_{M} is —C₁-C₆ alkylene-O—R_{S} (e.g., —C(CH₃)₂—CH₂—OMe); —C₁-C₆ alkylene-C(O)OR_{S} (e.g., —C(CH₃)₂—C(O)OMe); —C₁-C₆ alkylene-N(R_{S})C(O)OR_{S}′ (e.g., —C(CH₃)₂—CH₂—NHC(O)OCH₃); or —C₁-C₆ alkylene-P(O)(OR_{S})₂ (e.g., —CH₂—P(O)(OEt)₂). Also more preferably R_{M} is C₃-C₆ carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, —C(O)OR_{S}, or —N(R_{S}R_{S}′). For example R_{M} is cycloalkyl (e.g., cyclopropyl, 2,2-dichloro-1-methylcycloprop-1-yl, cyclohexyl), phenyl, heterocyclyl (e.g., morpholin-4-yl, 1,1-dioxidothiomorpholin-4-yl, 4-methylpiperazin-1-yl, 4-methoxycarbonylpiperazin-1-yl, pyrrolidin-1-yl, piperidin-1-yl, 4-methylpiperidin-1-yl, 3,5-dimethylpiperidin-1-yl, 4,4-difluoropiperidin-1-yl, tetrahydropyran-4-yl, pyridinyl, pyridin-3-yl, 6-(dimethylamino)pyridin-3-yl). Highly preferably, R_{M} is C₁-C₆alkyl which is optionally substituted with one or more substituents selected from halogen, hydroxy, mercapto, amino or carboxy (e.g., tert-butyl, CF₃).

More preferably, D is C₅-C₆ carbocycle, 5- to 6-membered heterocycle or 6- to 12-membered bicycle and is substituted with J and optionally substituted with one or more R_{A}, wherein J is C₃-C₆carbocycle, 3- to 6-membered heterocycle or 6- to 12-membered bicycle and is optionally substituted with one or more R_{A}. Preferably, J is substituted with a C₃-C₆ carbocycle or 3- to 6-membered heterocycle, wherein said C₃-C₆carbocycle or 3- to 6-membered heterocycle is independently optionally substituted with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆haloalkyl, C₂-C₆ haloalkenyl, C₂-C₆ haloalkynyl, C(O)OR_{S} or —N(R_{S}R_{S}′), and J can also be optionally substituted with one or more R_{A}. Also preferably, D is C₅-C₆carbocycle or 5- to 6-membered heterocycle and is substituted with J and optionally substituted with one or more R_{A}, and J is C₃-C₆ carbocycle or 3- to 6-membered heterocycle and is optionally substituted with one or more R_{A}, and preferably, J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C(O)OR_{S} or —N(R_{S}R_{S}′). Also preferably, D is C₅-C₆carbocycle or 5- to 6-membered heterocycle and is substituted with J and optionally substituted with one or more R_{A}, and J is 6- to 12-membered bicycle (e.g., a 7- to 12-membered fused, bridged or sipro bicycle comprising a nitrogen ring atom through which J is covalently attached to D) and is optionally substituted with one or more R_{A}. More preferably, D is phenyl and is substituted with J and optionally substituted with one or more R_{A}, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C(O)OR_{S} or —N(R_{S}R_{S}′). Highly preferably, D is

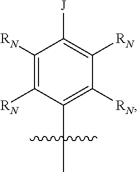

wherein each R_{N} is independently selected from R_{D} and preferably is hydrogen or halogen, and J is C₃-C₆carbocycle, 3- to 6-membered heterocycle or 6- to 12-membered bicycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C(O)OR_{S} or —N(R_{S}R_{S}′). Also preferably, D is

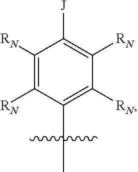

wherein each R_{N} is independently selected from R_{D} and preferably is hydrogen or halogen, and J is C₃-C₆carbocycle and 3- to 6-membered heterocycle and is substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C(O)OR_{S} or —N(R_{S}R_{S}′), and J can also be optionally substituted with one or more R_{A}. Also preferably, D is

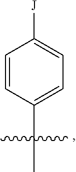

and J is C₃-C₆carbocycle or 3- to 6-membered heterocycle and is optionally substituted with one or more R_{A}, and preferably J is at least substituted with a C₃-C₆carbocycle or 3- to 6-membered heterocycle which is independently optionally substituted with one or more substituents selected from halogen, hydroxy, mercapto, amino, carboxy, nitro, oxo, phosphonoxy, phosphono, thioxo, formyl, cyano, C₁-C₆ alkyl, C₂-C₆alkenyl, C₂-C₆ alkynyl, C₁-C₆ haloalkyl, C₂-C₆ haloalkenyl, C₂-C₆ haloalkynyl, C(O)OR_{S} or —N(R_{S}R_{S}′).

The present invention also features -L₃-D, wherein:

D is C₃-C₁₂carbocycle or 3- to 12-membered heterocycle, and is optionally substituted with one or more R_{A}; or D is C₃-C₁₂carbocycle or 3- to 12-membered heterocycle which is substituted with J and optionally substituted with one or more R_{A}, where J is C₃-C₁₅carbocycle or 3- to 15-membered heterocycle (e.g., a 3- to 6-membered monocycle, a 6- to 12-membered fused, bridged or spiro bicycle, a 10- to 15-membered tricycle containing fused, bridged or spiro rings, or a 13- to 15-membered carbocycle or heterocycle) and is optionally substituted with one or more R_{A}, or J is —SF₅; or D is hydrogen or R_{A}; R_{A} and J are as defined herein;

R_{E} is independently selected at each occurrence from —O—R_{S}, —S—R₅, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, ═C(R_{S}R_{S}′), or —C(O)N(R_{S})C(O)—R_{S}′; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₁₂carbocycle or 3- to 12-membered heterocycle (e.g., 7- or 12-membered carbocycle or heterocycle), each of which is independently optionally substituted at each occurrence with one or more substituents selected from halogen, hydroxy, mercapto, amino, carboxy, nitro, oxo, phosphonoxy, phosphono, thioxo, formyl, cyano, trimethylsilyl, C₁-C₆alkyl, C₂-C₆alkenyl, C₂-C₆alkynyl, C₁-C₆haloalkyl, C₂-C₆haloalkenyl, C₂-C₆haloalkynyl, —O—R_{S}, —S—R_{S}, —C(O)R_{S}, —C(O)OR_{S}, or —N(R_{S}R_{S}′).

In one embodiment, D is a C₅-C₆ carbocycle or 5- to 6-membered heterocycle (e.g., phenyl), and is substituted with J and optionally substituted with one or more R_{A}. J is C₃-C₆carbocycle, 3- to 6-membered heterocycle, 6- to 12-membered bicycle, 10- to 15-membered tricycle, or 13- to 15-membered carbocycle/heterocycle, and J is optionally substituted with one or more R_{A}. Preferably, J is substituted with a C₃-C₆carbocycle, 3- to 6-membered heterocycle, 6- to 12-membered bicycle or 7- to 12-membered carbocycle/heterocycle, which is independently optionally substituted with one or more substituents selected from (1) halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —C(O)OR_{S} or —N(R_{S}R_{S}′), or (2) trimethylsilyl, —O—R_{S}, —S—R_{S}, —C(O)R_{S}; and J can also be optionally substituted with one or more R_{A}. Preferably, D is

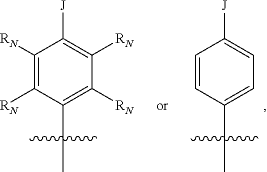

wherein J is as defined above, and each R_{N} is independently selected from R_{D} and preferably is hydrogen or halo such as F. L₁ and L₂ are each independently bond or C₁-C₆alkylene, and L₃ is bond, C₁-C₆alkylene or —C(O)—, and L₁, L₂, and L₃ are each independently optionally substituted with one or more R_{L}. Preferably, L₁, L₂, and L₃ are bond.

As used herein, R_{A} preferably is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl; or -L_{A}-O—R_{S}, -L_{A}-S—R_{S}, -L_{A}-C(O)R_{S}, -L_{A}-OC(O)R_{S}, -L_{A}-C(O)OR_{S}, -L_{A}-N(R_{S}R_{S}′), -L_{A}-S(O)R_{S}, -L_{A}-SO₂R_{S}, -L_{A}-C(O)N(R_{S}R_{S}′), -L_{A}-N(R_{S})C(O)R_{S}′, -L_{A}-N(R_{S})C(O)N(R_{S}′R_{S}″), -L_{A}-N(R_{S})SO₂R_{S}′, -L_{A}-SO₂N(R_{S}R_{S}′), -L_{A}-N(R_{S})SO₂N(R_{S}′R_{S}″), -L_{A}-N(R_{S})S(O)N(R_{S}′R_{S}″), -L_{A}-OS(O)—R_{S}, -L_{A}-OS(O)₂—R_{S}, -L_{A}-S(O)₂OR_{S}, -L_{A}-S(O)OR_{S}, -L_{A}-OC(O)OR_{S}, -L_{A}-N(R_{S})C(O)OR_{S}′, -L_{A}-OC(O)N(R_{S}R_{S}′), -L_{A}-N(R_{S})S(O)—R_{S}′, -L_{A}-S(O)N(R_{S}R_{S}′) or -L_{A}-C(O)N(R_{S})C(O)—R_{S}′, wherein L_{A} is bond, C₁-C₆alkylene, C₂-C₆alkenylene or C₂-C₆alkynylene.

More preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

Highly preferably, R_{A} is halogen, hydroxy, mercapto, amino, carboxy, nitro, oxo, phosphonoxy, phosphono, thioxo, cyano; or C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano.

L_{S}, L_{S}′ and L_{S}″ preferably are each independently selected at each occurrence from bond; or C₁-C₆alkylene, C₂-C₆alkenylene or C₂-C₆alkynylene.

According to another aspect of the invention, -L₃-D are defined as:

L₃ is bond or C₁-C₆ alkylene;

D is C₆-C₁₀carbocycle or 5- to 12-membered heterocycle, each of which is optionally R_{M} is independently selected at each occurrence from:
halogen, hydroxy, mercapto, amino, carboxy, nitro, oxo, phosphonoxy, phosphono, thioxo, cyano, SF₅, —N(R_{S}R_{S}′), —O—R_{S}, —OC(O)R_{S}, —OC(O)OR₅, —OC(O)N(R_{S}R_{S}′), —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —S(O)R_{S}, —SO₂R_{S}, —S(O)N(R_{S}R_{S}′), —SR₅, —Si(R_{S})₃, or —P(O)(OR_{S})₂;
C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —N(R_{S}R_{S}′), —O—R_{S}, —OC(O)R_{S}, —OC(O)OR_{S}, —OC(O)N(R_{S}R_{S}′), —C(O)R_{S}, —C(O)OR_{S}, —C(O)N(R_{S}R_{S}′), —N(R_{S})C(O)R_{S}′, —N(R_{S})C(O)OR_{S}′, —N(R_{S})SO₂R_{S}′, —S(O)R_{S}, —SO₂R_{S}, —S(O)N(R_{S}R_{S}′), —SR₅, or —P(O)(OR_{S})₂; or

G₂, wherein G₂ is a C₃-C₁₂ carbocycle or 3- to 12-membered heterocycle, each of which is independently optionally substituted at each occurrence with one or more R_{G2}, and each R_{G2} is independently selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl, C₂-C₆haloalkynyl, —O—R_{S}, —C(O)OR_{S}, —C(O)R_{S}, —N(R_{S}R_{S}′), or -L₄-G₃;

L₄ is a bond, C₁-C₆alkylene, C₂-C₆alkenylene, C₂-C₆alkynylene, —O—, —S—, —N(R_{B})—, —C(O)—, —S(O)₂—, —S(O)—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —N(R_{B})C(O)N(R_{B}′)—, —N(R_{B})SO₂N(R_{B}′)—, or —N(R_{B})S(O)N(R_{B}′)—;

G₃ is a C₃-C₁₂carbocycle or 3- to 12-membered heterocycle, and is optionally substituted with one or more R_{G3}; and

R_{G3} is each independently, at each occurrence, halogen, —C₁-C₆alkyl, —C(O)C₁-C₆alkyl, —C₁-C₆haloalkyl, —O—C₁-C₆alkyl, —O—C₁-C₆haloalkyl, C₃-C₆carbocycle, or 3- to 6-membered heterocycle. substituted with one or more R_{M};

R_{S}, R_{S}′ and R_{S}″ are each independently selected at each occurrence from hydrogen; C₁-C₆alkyl, C₂-C₆alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —O—C₁-C₆alkyl, —O—C₁-C₆haloalkyl, or 3- to 12-membered carbocycle or heterocycle; or 3- to 12-membered carbocycle or heterocycle; wherein each 3- to 12-membered carbocycle or heterocycle in R_{S}, R_{S}′ or R_{S}″ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl.

As described hereinabove for this aspect of the invention, D preferably is C₆-C₁₀carbocycle or 3 to 12-membered heterocycle optionally substituted by one or more R_{M}. Preferably, D is C₆-C₁₀ aryl (e.g., phenyl, naphthyl, indanyl), or 5- to 10-membered heteroaryl (pyridinyl, thiazolyl, 4,5,6,7-tetrahydrobenzo[d]thiazolyl, benzo[d]thiazolyl, indazolyl, benzo[d][1,3]dioxol-5-yl), and D is substituted with one or more R_{M}. For example, in certain embodiments D is preferably phenyl substituted by one or more R_{M}, wherein each R_{M} is independently halogen (e.g., fluoro, chloro, bromo); C₁-C₆alkyl (e.g., tert-butyl); C₁-C₆alkyl substituted with one or more halogen (e.g., CF₃); —O—R_{S} such as —O—C₁-C₆alkyl (e.g., —O—CH₂CH₃); or —O—C₁-C₆alkyl substituted at each occurrence with one or more halogen (e.g., —O—CF₃, —O—CH₂CHF₂) or —O—C₁-C₆alkyl (e.g., —O—CH₂CH₂OCH₃); —O—R_{S} (e.g., —O—C₁-C₆alkyl, such as —O—CH₂) substituted with 3- to 12-membered heterocycle (e.g., 3-ethyloxetan-3-yl, 1,3-dioxolan-4-yl); —O—R_{S} where R_{S} is an optionally substituted 3- to 12-membered carbocycle or heterocycle (e.g., cyclopentyl, cyclohexyl, phenyl, 1,3-dioxan-5-yl); —N(R_{S})C(O)R_{S}′ wherein R_{S} and R_{S}′ are each independently C₁-C₆alkyl (e.g., —N(t-Bu)C(O)Me); SF₅; —SO₂R_{S} wherein R_{S} is C₁-C₆alkyl (e.g., —SO₂Me); or C₃-C₁₂carbocycle (e.g., cyclopropyl, cyclohexyl, phenyl).

In certain embodiments of this aspect of the invention, D is preferably phenyl or pyridyl and is substituted by one or more R_{M} where one R_{M} is G₂. In certain embodiments where D is phenyl or pyridyl, D is substituted by G₂, G₂ is 3- to 12-membered heterocycle (e.g., pyridinyl, piperidinyl, pyrrolidinyl, azetidinyl, oxazolyl) and is optionally substituted with one or more halogen (e.g., fluoro, chloro), hydroxy, oxo, cyano, C₁-C₆alkyl (e.g., methyl), C₂-C₆alkenyl, C₂-C₆alkynyl, C₁-C₆haloalkyl (e.g., CF₃), C₂-C₆haloalkenyl, C₂-C₆haloalkynyl, —O—C₁-C₆alkyl (e.g., —O—CH₃), —C(O)OR_{S} (e.g., —C(O)OCH₃), —C(O)R_{S} (e.g., —C(O)CH₃), or —N(R_{S}R_{S}′); and D is further optionally substituted by one or more R_{M} where R_{M} is halogen (e.g., fluoro, chloro), C₁-C₆alkyl (e.g., methyl), C₁-C₆haloalkyl (e.g., CF₃), or —O—C₁-C₆ alkyl (e.g., —O—CH₃). In certain other embodiments D is phenyl or pyridyl and G₂ is, for example, a monocyclic 3-8 membered carbocycle or monocyclic 4-8 membered heterocycle substituted with L₄-G₃ and optionally substituted with one or more R_{G2} wherein L₄, G₃ and R_{G2} are as defined herein. L₄, for example is a bond, a C₁-C₆ alkylene (e.g., —CH₂—, —CH₂CH₂—, —CH₂CH₂CH₂—, etc.), —O—, or —S(O)₂—. G₃ is for example a C₃-C₁₂carbocycle optionally substituted with one or more R_{G3}. R_{G2} and R_{G3} are each independently at each occurrence halogen, —C(O)C₁-C₆alkyl, —C₁-C₆alkyl, —C₁-C₆haloalkyl, —O—C₁-C₆alkyl, or —O—C₁-C₆haloalkyl. In certain embodiments G₂ is

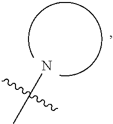 [SMILES: *N]

wherein

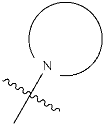 [SMILES: *N]

is a monocyclic 4-8 membered nitrogen-containing heterocycle (e.g., azetidinyl, pyrrolidinyl, piperidinyl, piperazinyl) attached to the parent molecular moiety through a nitrogen atom and substituted with one or two L₄-G₃ and optionally substituted with one or more R_{G2}. Thus, in certain embodiments where L₄ is a bond G₂ is

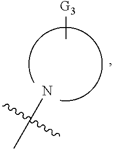

where

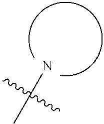 [SMILES: *N]

is optionally substituted with R_{G2} and G₄₂ is optionally substituted with R_{G3}. Thus.

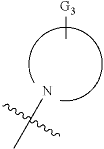

can be, for example, 3-phenylazetidin-1-yl, 3-phenylpyrrolidin-1-yl, 4-phenylpiperazin-1-yl, 4-phenylpiperidin-1-yl, 4-phenyl-3,6-dihydropyridin-1 (2H)-yl, 4,4-diphenylpiperidin-1-yl, 4-acetyl-4-phenylpiperidin-1-yl, 4-(4-methoxyphenyl)piperidin-1-yl, 4-(4-fluorophenyl)piperidin-1-yl, or 3-phenylpiperidin-1-yl, and wherein D can be further optionally substituted with one or more R_{M} (e.g., fluoro, chloro, methyl, methoxy).

In certain other embodiments of this aspect of the invention, L₄ is a C₁-C₆ alkylene, —O—, or —S(O)₂—, and G₂ is

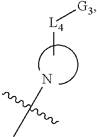

where

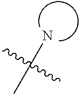 [SMILES: *N]

is as defined above and is optionally substituted with R_{G2} and G₃ is as defined above and is optionally substituted with R_{G3}. Thus,

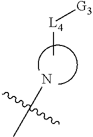

can be, for example, 4-tosylpiperazin-1-yl, 4-phenoxypiperidin-1-yl, 3-phenoxypyrrolidin-1-yl, 4-benzylpiperidin-1-yl, 4-phenethylpiperidin-1-yl, or 3-phenylpropyl)piperidin-1-yl.

In certain other embodiments of this aspect of the invention, D is phenyl or pyridyl, D is substituted by G₂ and G₂ is a spiro, bridged, or fused bicyclic carbocycle or heterocycle optionally substituted with L₄-G₃ and one or more R_{G2}, wherein D is optionally substituted with one or more R_{M} and R_{M}, L₄, G₃, and R_{G2} are as defined herein. In certain embodiments G₂ is

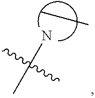 [SMILES: *N]

wherein

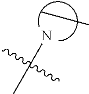 [SMILES: *N]

is a spiro, bridged, or fused bicyclic nitrogen-containing heterocycle (e.g., 3-azabicyclo[3.2.0]hept-3-yl, 2-azabicyclo[2.2.2]oct-2-yl, 6-azaspiro[2.5]oct-6-yl, octahydro-2H-isoindol-2-yl, 3-azaspiro[5.5]undec-3-yl, 1,3-dihydro-2H-isoindol-2-yl, 1,4-dioxa-8-azaspiro[4.5]dec-8-yl) attached to the parent molecular moiety through a nitrogen atom and optionally substituted with G₃ and one or more R_{G2}. Thus, G₂ is 3-azabicyclo[3.2.0]hept-3-yl, 2-azabicyclo[2.2.2]oct-2-yl, 6-azaspiro[2.5]oct-6-yl, octahydro-2H-isoindol-2-yl, 3-azaspiro[5.5]undec-3-yl, 1,3-dihydro-2H-isoindol-2-yl, or 1,4-dioxa-8-azaspiro[4.5]dec-8-yl; L₄ is a bond and D is optionally substituted with one or more R_{M} (e.g., fluoro, chloro, methyl, methoxy).

In certain embodiments of this aspect of the invention, D is

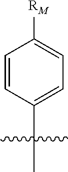

wherein R_{M} is as defined above in connection with Formula I_{E}, and D is optionally substituted by one or more additional R_{M}. For instance, where D is

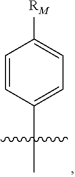

R_{M} can be fluoro, chloro, tert-butyl, —O—CH₂CH₃, —O—CF₃, —O—CH₂CHF₂, —O—CH₂CH₂OCH₃, —O—CH₂-(3-ethyloxetan-3-yl), —O—CH₂-(1,3-dioxolan-4-yl), —O-cyclopentyl, —O-cyclohexyl, —O-phenyl, —O-(1,3-dioxan-5-yl), cyclopropyl, cyclohexyl, phenyl, SF₅, —SO₂Me, or —N(t-Bu)C(O)Me and D can be optionally substituted by one or more additional R_{M} selected from the group consisting of halogen (e.g., fluoro, chloro) and C₁-C₆alkyl (e.g., methyl).

In certain embodiments of this aspect of the invention, D is

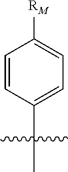

wherein R_{M} is fluoro, chloro, tert-butyl, —O—CH₂CH₃, —O—CF₃, —O—CH₂CHF₂, —O—CH₂CH₂OCH₃, SF₅, —SO₂Me, or —N(t-Bu)C(O)Me and D is optionally substituted by one or more additional R_{M} selected from the group consisting of halogen (e.g., fluoro, chloro) and C₁-C₆alkyl (e.g., methyl).

In certain embodiments of this aspect of the invention, D is

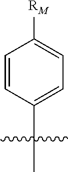

wherein R_{M} is cyclopropyl, cyclohexyl, or phenyl and D is optionally substituted by one or more additional R_{M} selected from the group consisting of halogen (e.g., fluoro, chloro) and C₁-C₆alkyl (e.g., methyl).

In certain embodiments of this aspect of the invention, D is

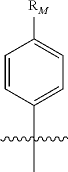

wherein R_{M} is —O—CH₂-(3-ethyloxetan-3-yl), —O—CH₂-(1,3-dioxolan-4-yl), —O-cyclopentyl, —O-cyclohexyl, —O-phenyl, or —O-(1,3-dioxan-5-yl) and D is optionally substituted by one or more additional R_{M} selected from the group consisting of halogen (e.g., fluoro, chloro) and C₁-C₆alkyl (e.g., methyl).

In certain embodiments of this aspect of the invention, D is

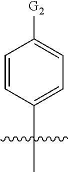

wherein G₂ is pyridinyl (e.g., pyridin-2-yl), piperidin-1-yl, 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, 3,3-dimethylazetidin-1-yl, or oxazolyl (e.g., 1,3-oxazol-2-yl) and D is optionally substituted by one or more additional R_{M} selected from the group consisting of halogen (e.g., fluoro, chloro) and C₁-C₆alkyl (e.g., methyl).

In another embodiment of this aspect of the invention, D is

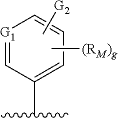

wherein G₁ is N, C—H, or C—R_{M}; G₂ is

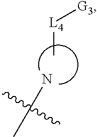

wherein

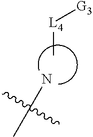

is a monocyclic 4-8 membered nitrogen-containing heterocycle (e.g., azetidinyl, pyrrolidinyl, piperidinyl) attached to the parent molecular moiety through a nitrogen atom and substituted by L₄-G₃ and optionally substituted with one or more R_{G2}; L₄ is a bond, C₁-C₆alkylene, —O—, or —S(O)₂—; G₃ is aryl (e.g., phenyl), cycloalkyl (e.g., cyclohexyl), or heterocycle (e.g., thienyl) wherein each G₃ is optionally substituted with one or more R_{G3}; R_{G2} and R_{G3} at each occurrence are each independently halogen, —C(O)C₁-C₆alkyl, —C₁-C₆alkyl, —C₁-C₆haloalkyl, —O—C₁-C₆alkyl, or —O—C₁-C₆haloalkyl; g is 0, 1, 2, or 3; and R_{M} is as defined above in connection with Formula I_{E}. In one group of compounds according to this embodiment, D is

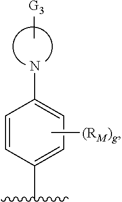

wherein G₃ is phenyl optionally substituted with one or two R_{G3}; g is 0, 1, or 2; R_{M} is each independently fluoro, chloro, methyl, methoxy, trifluoromethyl, or trifluoromethoxy; and

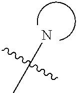 [SMILES: *N]

and R_{G3} are as defined above. In a further subgroup of compounds of this embodiment, D is

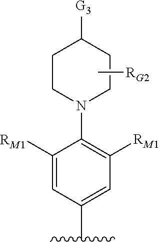

wherein G₃ is phenyl optionally substituted with one or two R_{G3}; R_{M1} is each independently hydrogen, fluoro, chloro, or methyl; and R_{G2} is an optional substituent as described herein. In another group of compounds according to this embodiment, D is

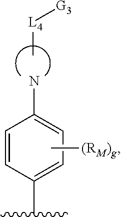

wherein L₄ is C₁-C₆ alkylene, —O—, or —S(O)₂—; G₃ is phenyl optionally substituted with one or two R_{G3}; g is 0, 1, or 2; R_{M} is each independently fluoro, chloro, methyl, methoxy, trifluoromethyl, or trifluoromethoxy; and

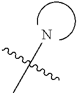 [SMILES: *N]

and R_{G3} are as defined above.

In yet another embodiment of this aspect of the invention, D is

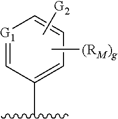

wherein G₁ is N, C—H, or C—R_{M}; G₂ is

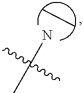

wherein

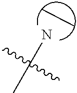

is a spiro, bridged, or fused bicyclic nitrogen-containing heterocycle (e.g., 3-azabicyclo[3.2.0]hept-3-yl, 2-azabicyclo[2.2.2]oct-2-yl, 6-azaspiro[2.5]oct-6-yl, octahydro-2H-isoindol-2-yl, 3-azaspiro[5.5]undec-3-yl, 1,3-dihydro-2H-isoindol-2-yl, 1,4-dioxa-8-azaspiro[4.5]dec-8-yl) attached to the parent molecular moiety through a nitrogen atom and optionally substituted with L₄-G₃ and one or more R_{G2}; L₄ is a bond, C₁-C₆ alkylene, —O—, or —S(O)₂—; G₃ is aryl (e.g., phenyl), cycloalkyl (e.g., cyclohexyl), or heterocycle (e.g., thienyl) wherein each G₃ is optionally substituted with one or more R_{G3}; R_{G2} and R_{G3} at each occurrence are each independently halogen, —C(O)C₁-C₆alkyl, —C₁-C₆alkyl, —C₁-C₆haloalkyl, —O—C₁-C₆alkyl, or —O—C₁-C₆haloalkyl; g is 0, 1, 2, or 3; and R_{M} is as defined above in connection with Formula I_{E}. In one group of compounds according to this embodiment, D is

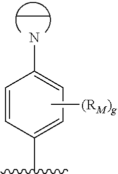

wherein g is 0, 1, or 2; R_{M} is each independently fluoro, chloro, methyl, methoxy, trifluoromethyl, or trifluoromethoxy; and

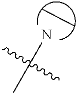

is as defined above. In a further subgroup of compounds D is

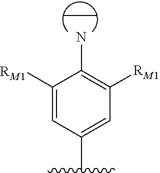

wherein R_{M1} is each independently hydrogen, fluoro, chloro, or methyl, and

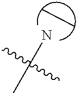

is as defined above (e.g., 3-azabicyclo[3.2.0]hept-3-yl, octahydro-2H-isoindol-2-yl, 2-azabicyclo[2.2.2]oct-2-yl, 6-azaspiro[2.5]oct-6-yl, 3-azaspiro[5.5]undec-3-yl, 1,3-dihydro-2H-isoindol-2-yl, 1,4-dioxa-8-azaspiro[4.5]dec-8-yl).

In still another embodiment of this aspect of the invention, D is

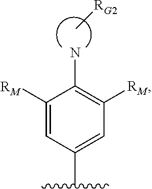

wherein

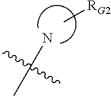

is a monocyclic 4-8 membered nitrogen-containing heterocycle (e.g., azetidinyl, pyrrolidinyl, piperidinyl) substituted with one or more R_{G2}, wherein R_{G2} at each occurrence is each independently halogen, —C(O)C₁-C₆alkyl, —C₁-C₆alkyl, —C₁-C₆haloalkyl, —O—C₁-C₆alkyl, or —O—C₁-C₆haloalkyl; and R_{M} is each independently halogen, —C₁-C₆alkyl, —C₁-C₆haloalkyl, —O—C₁-C₆alkyl, or —O—C₁-C₆haloalkyl. In one group of compounds according to this embodiment,

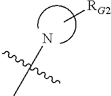

is azetidinyl, pyrrolidinyl, or piperidinyl substituted with one or two R_{G2}, wherein R_{G2} at each occurrence is each independently methyl, ethyl, isopropyl, tert-butyl, fluoro, chloro, or trifluoromethyl; and R_{M} is each independently fluoro, chloro, or methyl. For example

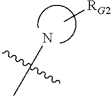

is 4,4-dimethylpiperidin-1-yl, 4,4-difluoropiperidin-1-yl, 2,6-dimethylpiperidin-1-yl, 4-(propan-2-yl)piperidin-1-yl, 4-fluoropiperidin-1-yl, 3,5-dimethylpiperidin-1-yl, 4-(trifluoromethyl)piperidin-1-yl, 4-methylpiperidin-1-yl, 4-tert-butylpiperidin-1-yl, 2-oxopiperidin-1-yl, or 3,3-dimethylazetidin-1-yl.

Non-limited examples of D in -L₃-D include:

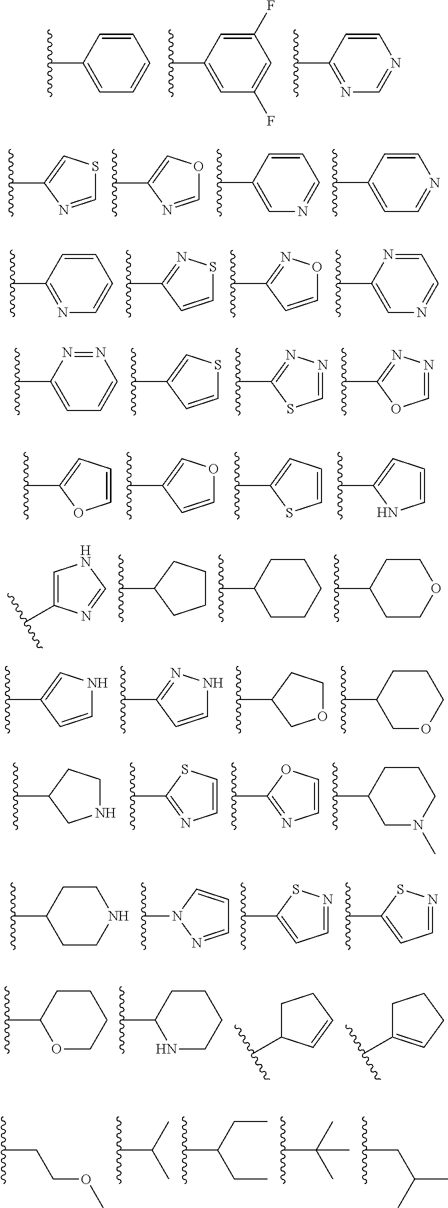

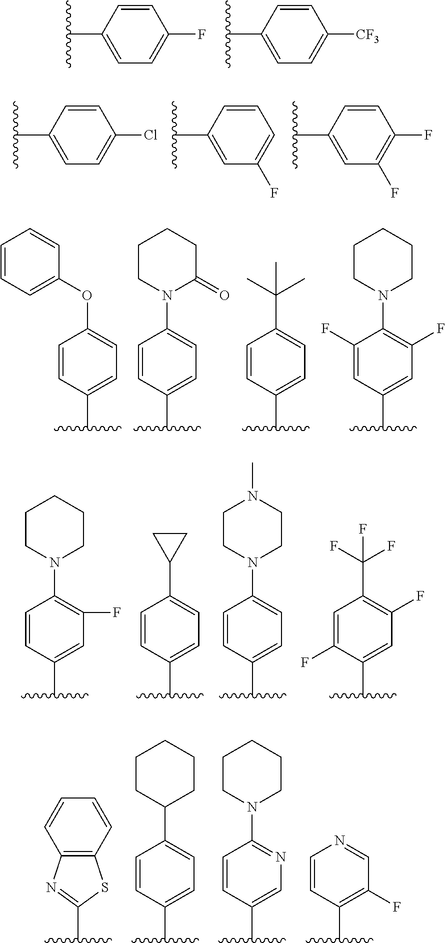

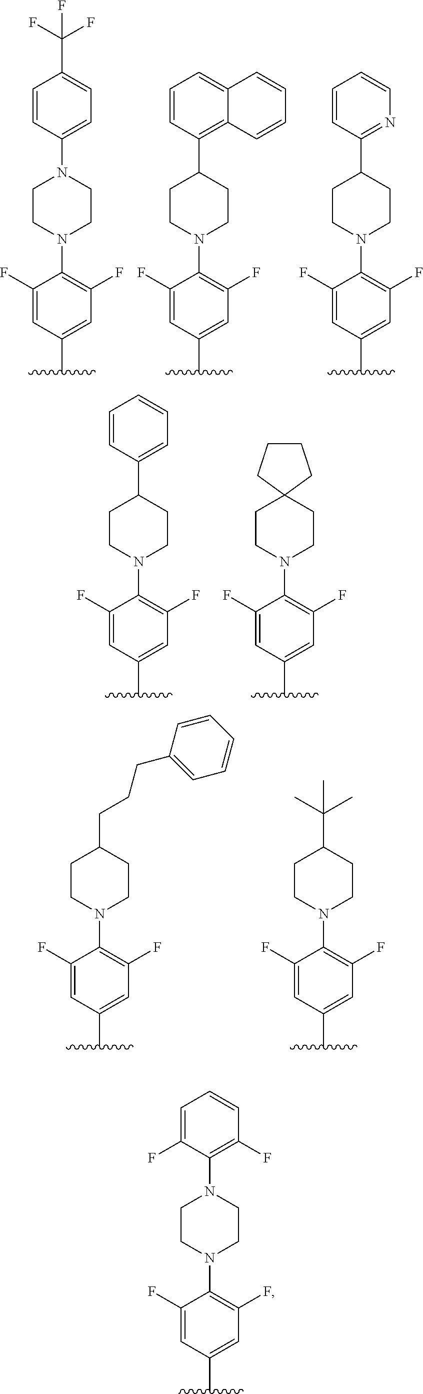

wherein L₃ is preferably bond.

The term “alkenyl” as used in connection with the definition of -L-E or -L₃-D means a straight or branched hydrocarbyl chain containing one or more double bonds. Each carbon-carbon double bond may have either cis or trans geometry within the alkenyl moiety, relative to groups substituted on the double bond carbons. Non-limiting examples of alkenyl groups include ethenyl (vinyl), 2-propenyl, 3-propenyl, 1,4-pentadienyl, 1,4-butadienyl, 1-butenyl, 2-butenyl, and 3-butenyl.

The term “alkenylene” as used in connection with the definition of -L-E or -L₃-D refers to a divalent unsaturated hydrocarbyl chain which may be linear or branched and which has at least one carbon-carbon double bond. Non-limiting examples of alkenylene groups include —C(H)═C(H)—, —C(H)═C(H)—CH₂—, —C(H)═C(H)—CH₂—CH₂—, —CH₂—C(H)═C(H)—CH₂—, —C(H)═C(H)—CH(CH₃)—, and —CH₂—C(H)═C(H)—CH(CH₂CH₃)—.

The term “alkyl” as used in connection with the definition of -L-E or -L₃-D means a straight or branched saturated hydrocarbyl chain. Non-limiting examples of alkyl groups include methyl, ethyl, n-propyl, isopropyl, n-butyl, isobutyl, sec-butyl, t-butyl, pentyl, iso-amyl, and hexyl.

The term “alkylene” as used in connection with the definition of -L-E or -L₃-D denotes a divalent saturated hydrocarbyl chain which may be linear or branched. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkynyl” as used in connection with the definition of -L-E or -L₃-D means a straight or branched hydrocarbyl chain containing one or more triple bonds. Non-limiting examples of alkynyl include ethynyl, 1-propynyl, 2-propynyl, 3-propynyl, decynyl, 1-butynyl, 2-butynyl, and 3-butynyl.

The term “alkynylene” as used in connection with the definition of -L-E or -L₃-D refers to a divalent unsaturated hydrocarbon group which may be linear or branched and which has at least one carbon-carbon triple bonds. Representative alkynylene groups include, by way of example, —C≡C—, —C≡C—CH₂—, —C≡C—CH₂—CH₂—, —CH₂—C≡C—CH₂—, —C≡C—CH(CH₃)—, and —CH₂—C≡C—CH(CH₂CH₃)—.

The term “carbocycle” or “carbocyclic” or “carbocyclyl” as used in connection with the definition of -L-E or -L₃-D refers to a saturated (e.g., “cycloalkyl”), partially saturated (e.g., “cycloalkenyl” or “cycloalkynyl”) dr completely unsaturated (e.g., “aryl”) ring system containing zero heteroatom ring atom. “Ring atoms” or “ring members” are the atoms bound together to form the ring or rings. A carbocyclyl may be, without limitation, a single ring, two fused rings, or bridged or spiro rings. A substituted carbocyclyl may have either cis or trans geometry. Representative examples of carbocyclyl groups include, but are not limited to, cyclopropyl, cyclobutyl, cyclopentyl, cyclohexyl, cycloheptyl, cyclooctyl, cyclopentenyl, cyclopentadienyl, cyclohexadienyl, adamantyl, decahydro-naphthalenyl, octahydro-indenyl, cyclohexenyl, phenyl, naphthyl, indanyl, 1,2,3,4-tetrahydro-naphthyl, indenyl, isoindenyl, decalinyl, and norpinanyl. A carbocycle group can be attached to the parent molecular moiety through any substitutable carbon ring atom.

The term “carbocyclylalkyl” as used in connection with the definition of -L-E or -L₃-D refers to a carbocyclyl group appended to the parent molecular moiety through an alkylene group. For instance, C₃-C₆carbocyclylC₁-C₆alkyl refers to a C₃-C₆carbocyclyl group appended to the parent molecular moiety through C₁-C₆alkylene.

The term “cycloalkenyl” as used in connection with the definition of -L-E or -L₃-D as used in connection with the definition of -L-E or -L₃-D refers to a non-aromatic, partially unsaturated carbocyclyl moiety having zero heteroatom ring member. Representative examples of cycloalkenyl groups include, but are not limited to, cyclobutenyl, cyclopentenyl, cyclohexenyl, and octahydronaphthalenyl.

The term “cycloalkyl” as used in connection with the definition of -L-E or -L₃-D refers to a saturated carbocyclyl group containing zero heteroatom ring member. Non-limiting examples of cycloalkyls include cyclopropyl, cyclobutyl, cyclopentyl, cyclohexyl, cycloheptyl, cyclooctyl, decalinyl and norpinanyl.

The prefix “halo” as used in connection with the definition of -L-E or -L₃-D indicates that the substituent to which the prefix is attached is substituted with one or more independently selected halogen radicals. For example, “C₁-C₆ haloalkyl” means a C₁-C₆ alkyl substituent wherein one or more hydrogen atoms are replaced with independently selected halogen radicals. Non-limiting examples of C₁-C₆haloalkyl include chloromethyl, 1-bromoethyl, fluoromethyl, difluoromethyl, trifluoromethyl, and 1,1,1-trifluoroethyl. It should be recognized that if a substituent is substituted by more than one halogen radical, those halogen radicals may be identical or different (unless otherwise stated).

The term “heterocycle” or “heterocyclo” or “heterocyclyl” as used in connection with the definition of -L-E or -L₃-D refers to a saturated (e.g., “heterocycloalkyl”), partially unsaturated (e.g., “heterocycloalkenyl” or “heterocycloalkynyl”) or completely unsaturated (e.g., “heteroaryl”) ring system where at least one of the ring atoms is a heteroatom (i.e., nitrogen, oxygen or sulfur), with the remaining ring atoms being independently selected from the group consisting of carbon, nitrogen, oxygen and sulfur. A heterocycle may be, without limitation, a single ring, two fused rings, or bridged or spiro rings. A heterocycle group can be linked to the parent molecular moiety via any substitutable carbon or nitrogen atom(s) in the group.

A heterocyclyl may be, without limitation, a monocycle which contains a single ring. Non-limiting examples of monocycles include furanyl, dihydrofuranyl, tetrahydrofuranyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), and 1,3,4-oxadiazolyl), oxatriazolyl (including 1,2,3,4-oxatriazolyl and 1,2,3,5-oxatriazolyl), dioxazolyl (including 1,2,3-dioxazolyl, 1,2,4-dioxazolyl, 1,3,2-dioxazolyl, and 1,3,4-dioxazolyl), oxathiolanyl, pyranyl (including 1,2-pyranyl and 1,4-pyranyl), dihydropyranyl, pyridinyl, piperidinyl, diazinyl (including pyridazinyl (also known as “1,2-diazinyl”), pyrimidinyl (also known as “1,3-diazinyl”), and pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl), oxazinyl (including 1,2,3-oxazinyl, 1,3,2-oxazinyl, 1,3,6-oxazinyl (also known as “pentoxazolyl”), 1,2,6-oxazinyl, and 1,4-oxazinyl), isoxazinyl (including o-isoxazinyl and p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl and 1,3,5,2-oxadiazinyl), morpholinyl, azepinyl, oxepinyl, thiepinyl, and diazepinyl.

A heterocyclyl may also be, without limitation, a bicycle containing two fused rings, such as, for example, naphthyridinyl (including [1,8]naphthyridinyl, and [1,6]naphthyridinyl), thiazolpyrimidinyl, thienopyrimidinyl, pyrimidopyrimidinyl, pyridopyrimidinyl, pyrazolopyrimidinyl, indolizinyl, pyrindinyl, pyranopyrrolyl, 4H-quinolizinyl, purinyl, pyridopyridinyl (including pyrido[3,4-b]-pyridinyl, pyrido[3,2-b]-pyridinyl, and pyrido[4,3-b]-pyridinyl), pyridopyrimidine, and pteridinyl. Other non-limiting examples of fused-ring heterocycles include benzo-fused heterocyclyls, such as indolyl, isoindolyl, indoleninyl (also known as “pseudoindolyl”), isoindazolyl (also known as “benzpyrazolyl”), benzazinyl (including quinolinyl (also known as “l-benzazinyl”) and isoquinolinyl (also known as “2-benzazinyl”)), benzimidazolyl, phthalazinyl, quinoxalinyl, benzodiazinyl (including cinnolinyl (also known as “1,2-benzodiazinyl”) and quinazolinyl (also known as “1,3-benzodiazinyl”)), benzopyranyl (including “chromenyl” and “isochromenyl”), benzothiopyranyl (also known as “thiochromenyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, and “benzothiofuranyl”), isobenzothienyl (also known as “isobenzothiophenyl”, “isothionaphthenyl”, and “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, and 3,1,4-benzoxazinyl), benzisoxazinyl (including 1,2-benzisoxazinyl and 1,4-benzisoxazinyl), and tetrahydroisoquiriolinyl.

A heterocyclyl may comprise one or more sulfur atoms as ring members; and in some cases, the sulfur atom(s) is oxidized to SO or SO₂. The nitrogen heteroatom(s) in a heterocyclyl may or may not be quaternized, and may or may not be oxidized to N-oxide. In addition, the nitrogen heteroatom(s) may or may not be N-protected.

The number of carbon atoms in a hydrocarbyl moiety can be indicated by the prefix “Cₓ-C_{y},” where x is the minimum and y is the maximum number of carbon atoms in the moiety. Thus, for example, “C₁-C₆alkyl” refers to an alkyl substituent containing from 1 to 6 carbon atoms. Illustrating further, C₃-C₆carbocycle means a carbocycle containing from 3 to 6 carbon ring atoms. A prefix attached to a multiple-component substituent only applies to the first component that immediately follows the prefix. To illustrate, the term “carbocyclylalkyl” contains two components: carbocyclyl and alkyl. Thus, for example, C₃-C₆ carbocyclyl C₁-C₆ alkyl refers to a C₃-C₆ carbocyclyl appended to the parent molecular moiety through a C₁-C₆ alkyl group.

Unless otherwise specified, when a moiety links two other elements in a depicted chemical structure, the leftmost-described component of the moiety is bound to the left element in the depicted structure, and the rightmost-described component of the moiety is bound to the right element in the depicted structure. To illustrate, if the chemical structure is -L-L_{S}-R_{E} and L_{S} is C₁-C₆ alkylene, then the chemical structure is -L-C₁-C₆ alkylene-R_{E}.

If a moiety in a depicted structure is a bond, then the element left to the moiety is joined directly to the element right to the linking element via a covalent bond. For example, if a chemical structure is depicted as -L-L_{S}-R_{E} and L_{S} is selected as bond, then the chemical structure will be -L-R_{E}. If two or more adjacent moieties in a depicted structure are bonds, then the element left to these moieties is joined directly to the element right to these linking elements via a covalent bond.

When a chemical formula is used to describe a moiety, the dash(s) indicates the portion of the moiety that has the free valence(s).

If a moiety is described as being “optionally substituted”, the moiety may be either substituted or unsubstituted. If a moiety is described as being optionally substituted with up to a particular number of non-hydrogen radicals, that moiety may be either unsubstituted, or substituted by up to that particular number of non-hydrogen radicals or by up to the maximum number of substitutable positions on the moiety, whichever is less. Thus, for example, if a moiety is described as a heterocycle optionally substituted with up to three non-hydrogen radicals, then any heterocycle with less than three substitutable positions will be optionally substituted by up to only as many non-hydrogen radicals as the heterocycle has substitutable positions. To illustrate, tetrazolyl (which has only one substitutable position) will be optionally substituted with up to one non-hydrogen radical. To illustrate further, if an amino nitrogen is described as being optionally substituted with up to two non-hydrogen radicals, then a primary amino nitrogen will be optionally substituted with up to two non-hydrogen radicals, whereas a secondary amino nitrogen will be optionally substituted with up to only one non-hydrogen radical.

In an embodiment, this aspect of the present invention relates to compounds of Formula (IIa), or a pharmaceutically acceptable salt thereof:

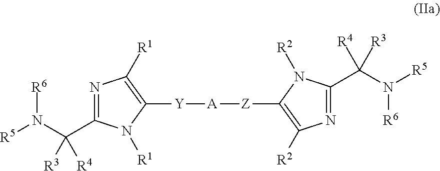

wherein A, Y, Z, R¹, R², R³, R⁴, R⁵ and R⁶ are as previously defined above.

In still yet another embodiment, this aspect of the present invention relates to compounds of Formula (IIb), or a pharmaceutically acceptable salt thereof:

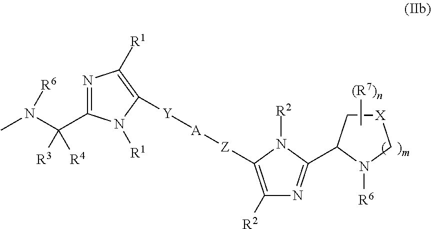

wherein A, Y, Z, m, n, R¹, R², R³, R⁴, R⁵, R⁶ and R⁷ are as previously defined above.

In still another embodiment, this aspect of the present invention relates to compounds of Formula (IIc), or a pharmaceutically acceptable salt thereof:

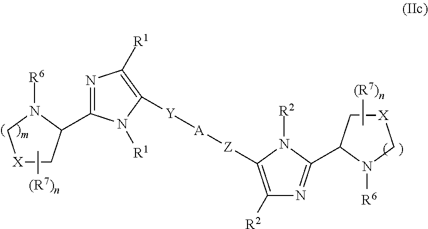

wherein A, Y, Z, m, n, R¹, R², R⁶ and R⁷ are as previously defined above.

In still another embodiment, this aspect the present invention relates to compounds of Formula (IId), or a pharmaceutically acceptable salt thereof:

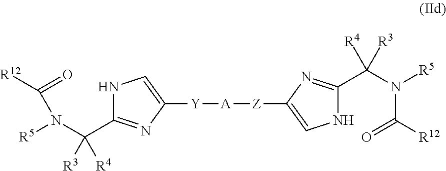

wherein A, Y, Z, R³, R⁴, R⁵ and R¹² are as previously defined above.

In still another embodiment, this aspect of the present invention relates to compounds of Formula (IIe), or a pharmaceutically acceptable salt thereof:

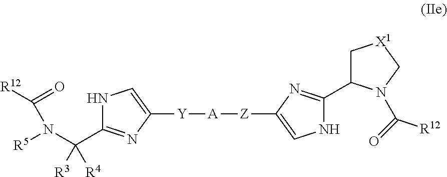

wherein A, Y, Z, R³, R⁴, R⁵ and R¹² are as previously defined above and X, at each occurrence is independently CH₂, CHF, CH(OH), or CF₂.

In still another embodiment, this aspect of the present invention relates to compounds of Formula (IIf), or a pharmaceutically acceptable salt thereof:

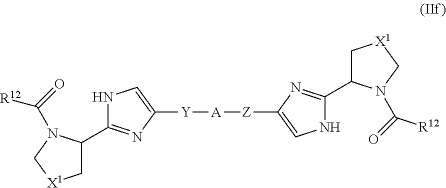

wherein A, Y, Z, X¹ and R¹² are as previously defined above.

In still another embodiment, this aspect of the present invention relates to compounds of Formula (IIf), wherein R¹² is C₁-C₈ alkyl optionally substituted with amino, hydroxy, phenyl, protected amino, or O(C₁-C₄ alkyl); or a pharmaceutically acceptable salt thereof.

In still yet another embodiment, this aspect of the present invention is the absolute stereochemistry of the pyrrolidine and 2-imi-dazolylmethylamine moiety, is represented by Formulae (IIg-1, IIg-2 and IIg-3):

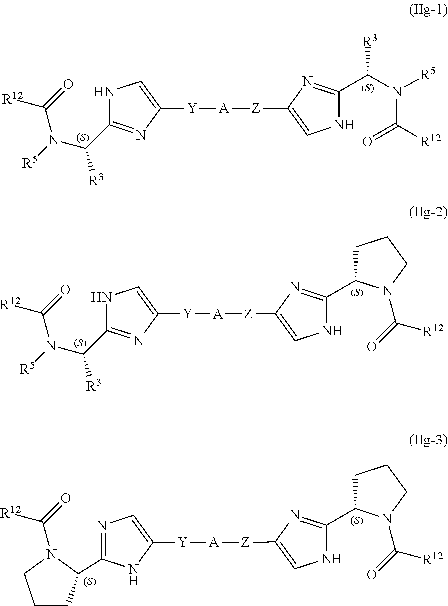

wherein A, Y, Z, R³, R⁵, and R¹² are as previously defined above.

In still yet another embodiment, this aspect of the present invention relates to compounds of Formula (IIh), or a pharmaceutically acceptable salt thereof:

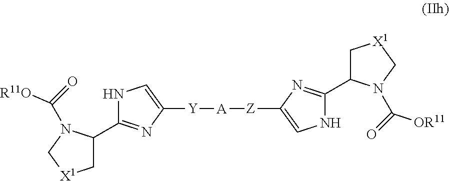

wherein A, Y, Z, X¹ and R¹¹ are as previously defined above.

In still yet another embodiment, this aspect of the present invention relates to compounds of Formula (IIi), or a pharmaceutically acceptable salt thereof:

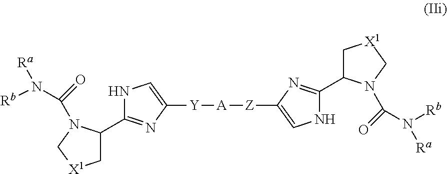

wherein, A, Y, Z, R^{a} and R^{b} are as previously defined above.

In still yet another embodiment, this aspect of the present invention relates to compounds of Formula (IIj), or a pharmaceutically acceptable salt thereof:

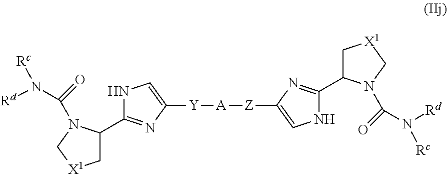

wherein, A, Y, X¹, R^{c} and R^{d} are as previously defined above.

In still yet another embodiment, this aspect of the present invention relates to compounds of Formula (IIk), or a pharmaceutically acceptable salt thereof:

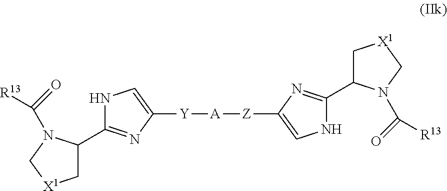

wherein A, Y, Z, X¹, and R³ are as previously defined above.

The present invention also contemplates compounds of Formula II and IIa-IIe in US Patent Publication 2010/0226883 or pharmaceutically acceptable salts thereof, wherein Y, A or Z, if or when present, can be substituted with with -L-E or -L₃-D as defined above herein. The present invention further contemplates compounds of Formula IIIa-IIIk in US Patent Publication 2010/0226883, wherein Y, A or Z, if or when present, can be substituted with with -L-E or -L₃-D as defined above herein.

Except for the definitions provided for -L-E or -L₃-D provided previously herein, the remaining substitutents in the compound having above Formula II as well as other formulae described above are to be interpreted according to the meaning provided for the substitutents in US Patent Publication 2010/0226883, the contents of which are herein incorporated by reference.

Methods for making compounds of Formula II as well as other formulae described above are described in US Patent Publication 2010/0226883 (see the description of the compounds having Formula I) and U.S. application Ser. No. 12/959,941 filed on Dec. 3, 2010, the contents of which are herein each incorporated by reference.

A second aspect, the present invention relates to compounds having the structure of below Formula (V) or pharmaceutically acceptable salts thereof:

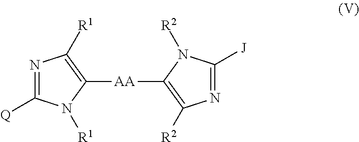

wherein:
AA is an (a) aromatic group selected from the group consisting of:
i. a bicyclic aryl or bicyclic heteroaryl, wherein the rings of said bicyclic ring system are fused or covalently attached, wherein each imidazole moiety is attached to a different ring of said bicyclic ring system and wherein at least one ring of said bicyclic is a 5- or 7-membered ring;
ii. a tricyclic aryl or tricyclic heteroaryl, wherein the rings of said tricyclic ring system are fused, covalently attached or a combination thereof, and wherein each imidazole moiety is attached to a different ring of said tricyclic; and

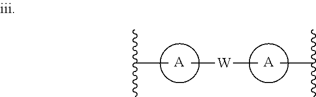

each A is independently a 5-7 membered aryl or heteroaryl, wherein said aryl or heteroaryl are optionally substituted; or

wherein AA is substituted with -L-E or -L₃-D, wherein L, E, L₃ and D are defined as above in connection with the compounds of Formula II;

W is an aliphatic group containing zero to eight carbons; preferably selected from the group consisting of O, S, SO, SO₂, C(O), NR¹¹, optionally substituted C₁-C₄ alkyl, optionally substituted C₂-C₄alkenyl and C₁-C₄ alkoxy;

R¹¹ at each occurrence is independently hydrogen or optionally substituted C₁-C₈ alkyl;

R¹ and R² at each occurrence are each independently selected from the group consisting of: halogen, cyano, optionally substituted C₁-C₄ alkyl, —O—R¹¹, —NR^{a}R^{b}, —C(O)R¹¹, —CO₂R¹¹, and —C(O)NR^{a}R^{b}, preferably halogen and optionally substituted C₁-C₄ alkyl; wherein at least one of each of R¹ and R² is substituted with -L-E or -L₃-D as defined above in connection with the compounds of Formula II;

R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;

Q and J are each independently selected from:

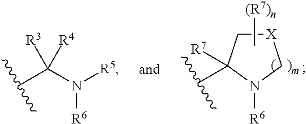

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; preferably hydrogen or optionally substituted C₁-C₄ alkyl; or alternatively, R³ and R⁴ can be taken together with the carbon atom to which they are attached to form optionally substituted C₃-C₈ cycloalkyl or optionally substituted heterocyclic;

R⁵ at each occurrence is independently hydrogen, optionally substituted C₁-C₈ alkyl, or optionally substituted C₃-C₈ cycloalkyl; preferably hydrogen or optionally substituted C₁-C₄ alkyl;

R⁶ at each occurrence is independently, selected from the group consisting of —C(O)—R¹², —C(O)—C(O)—R¹², —S(O)₂—R¹², and —C(S)—R², preferably —C(O)—R², more preferably an optionally substituted amino acid acyl;

R¹² at each occurrence is independently selected from the group consisting of: —O—R¹¹, —NR^{a}—R^{b}, —R¹³, —NR^{c}R^{d}, preferably optionally substituted C₁-C₈ alkyl and —O—R¹¹;

R¹³ at each occurrence is independently selected from the group consisting of hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; preferably optionally substituted C₁-C₈ alkyl; more preferably C₁-C₈ alkyl optionally substituted with amino, hydroxy, optionally substituted phenyl, protected amino, or O(C₁-C₄ alkyl); and

R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R¹³)₂;

m is 0, 1, or 2, preferably 1;

n is 1, 2, 3, or 4, preferably 1 or 2;

X at each occurrence is independently selected from O, S, S(O), SO₂, and C(R⁷)₂, preferably CH₂or CHR⁷; provided that when m is 0, X is C(R⁷)₂; and

R⁷ at each occurrence is independently selected from the group consisting of hydrogen, halogen, cyano, —O—R¹¹, —NR^{a}R^{b}, optionally substituted aryl, optionally substituted heteroaryl, and optionally substituted —C₁-C₄ alkyl; preferably hydrogen, methyl or halogen; or two vicinal R⁷ groups can be taken together with the two adjacent atoms to which they are attached to form a fused, optionally substituted C₃-C₈ cycloalkyl or optionally substituted heterocyclic ring; preferably a fused, optionally substituted cyclopropyl; or alternatively two geminal R⁷ groups can be taken together with the carbon atom to which they are attached to form a spiro, optionally substituted C₃-C₈ cycloalkyl or optionally substituted heterocyclic ring; preferably a spiro, optionally substituted cyclopropyl.

The present invention also contemplates compounds of Formula 1-Ia-1-If, 1-Ig-1, 1-Ig-2, 1-Ig-3 and 1-Ih-1-Il and pharmaceutically acceptable salts thereof as described in WO 2010/096462 wherein “L” in each of these formulae is substituted with -L-E or -L₃-D as defined herein. Likewise, the present invention contemplates compounds of Formula 1-IIa, 1-IIb, 1-IIc, 1-IId, 1-IIIa, 1-IIIb, 1-IIIc, 1-IIId, 1-IIIe, 1-IIIf, 1-IIIg, 1-IIIh, 1-IVa, 1-IVb, 1-IVc, 1-IVd, 1-IVe, 1-IVf, 1-IVg, 1-IVh, 1-IVi, or 1-IVj and pharmaceutically acceptable salts thereof as described in WO 2010/096462, wherein the moiety between the immidazoles in each formula (e.g,

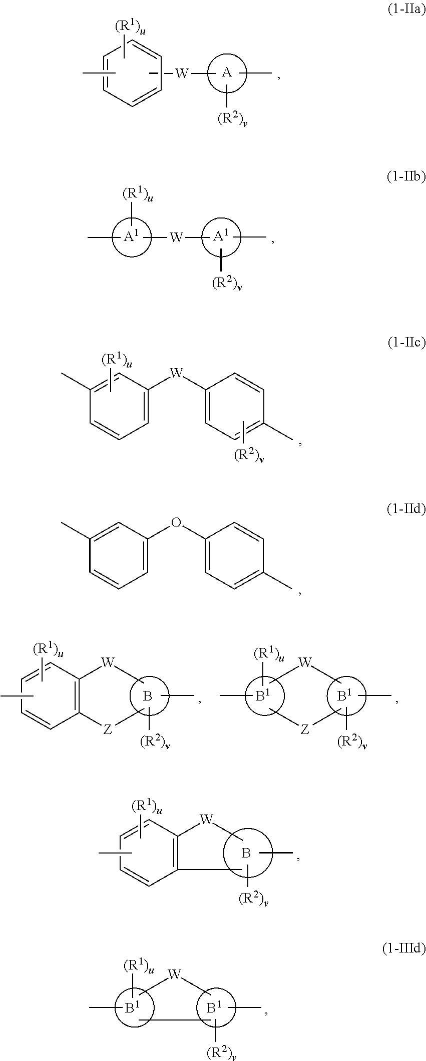

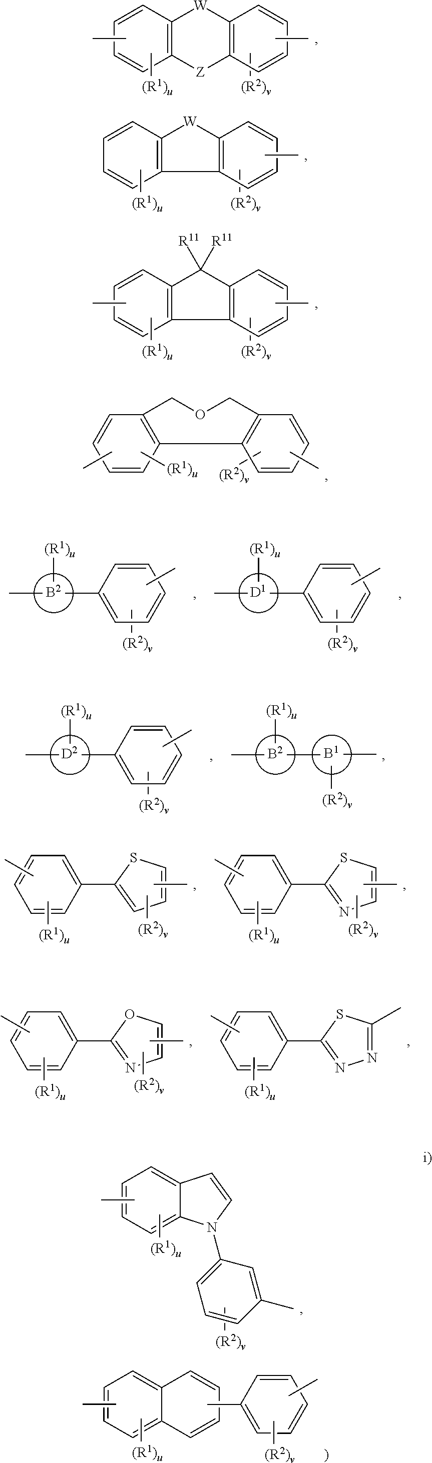

is substituted with -L-E or -L₃-D.

Except for the definitions provided for -L-E or -L-D provided previously herein, the remaining substitutents in the compound having above Formula V as well as other formulae described above are to be interpreted according to the meaning provided for the substitutents in the compound having Formula 1-I as described in WO 2010/096462 beginning on page 65 (with the section starting “Definitions”), the contents of which are herein incorporated by reference.

Methods for making compounds of Formula V as well as other formulae described above are described in WO 2010/096462 (See, compounds having the Formula 1-I) and U.S. application Ser. No. 12/959,941 filed on Dec. 3, 2010, the contents of which are herein each incorporated by reference.

In a third aspect, the present invention relates to compounds having the structure of below Formula (VI) or pharmaceutically acceptable salts thereof:

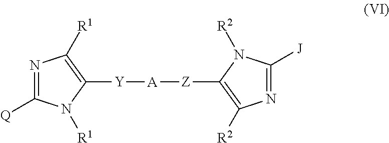

A is (a) absent; or a (b) a cyclic group independently selected from aryl, heteroaryl, heterocyclic, C₃-C₈ cycloalkyl, and C₃-C₈ cycloalkenyl, each optionally substituted; wherein A, if or when present, preferably is substituted with -L-E or -L₃-D, wherein L, E, L₃ and D are defined above in connection with the compounds of Formula II;

Y is (a) absent; or (b) an optionally substituted aliphatic group selected from the group consisting of O, S, N(R¹¹), C₁-C₈ alkyl, C₂-C₈ alkenyl, C₂-C₈ alkynyl, C₃-C₈ cycloalkyl, C₃-C₈ cycloalkenyl, and heterocyclic, each optionally substituted; or a combination thereof; wherein Y, if or when present, is optionally substituted with -L-E or -L₃-D, wherein L, E, L₃ and D are defined above in connection with the compounds of Formula II;

Z is (a) absent; or (b) an optionally substituted linear aliphatic group; optionally Z contains a group selected from O, N(R¹¹), C(O), S(O)₂, C(O)O, C(O)N(R¹¹), OC(O)O, OC(O)N(R¹¹), S(O)₂N(R¹¹), N(R¹¹)C(O)N(R¹¹), N(R¹¹)C(O)C(O)N(R¹¹), N(R¹¹)S(O)₂N(R¹¹), C(O)N(R¹)S(O)₂ and C(O)N(R¹¹)S(O)₂N(R¹¹); wherein Z, if or when present, is optionally independently substituted with -L-E or -L₃-D, wherein L, E, L₃ and D are defined above in connection with the compounds of Formula II;

wherein preferably at least two of A, Y, and Z are present; wherein at least one of A, Y or Z is substituted with -L-E or -L₃-D;

R¹¹ at each occurrence is independently hydrogen or optionally substituted C₁-C₈ alkyl;

R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —C(O)₂R¹¹, and —C(O)NR^{a}R^{b}; preferably hydrogen, halogen and optionally substituted C₁-C₄ alkyl; wherein at least one of R¹ and R² is substituted with -L-E or -L₃-D, wherein L, E, L₃ and D are defined above in connection with the compounds of Formula II;

R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;

Q and J are each independently selected from:

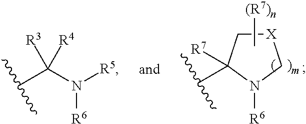

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; preferably hydrogen or optionally substituted C₁-C₄ alkyl; or alternatively, R³ and R⁴ can be taken together with the carbon atom to which they are attached to form optionally substituted C₃-C₈ cycloalkyl or optionally substituted heterocyclic;

R⁵ at each occurrence is independently hydrogen, optionally substituted C₁-C₈ alkyl, or optionally substituted C₃-C₈ cycloalkyl; preferably hydrogen or optionally substituted C₁-C₄ alkyl;

R⁶ at each occurrence is independently selected from the group consisting of —C(O)—R¹², —C(O)—C(O)—R¹², —S(O)₂—R¹², and —C(S)—R¹², preferably —C(O)—R², more preferably an optionally substituted amino acid acyl;

R¹² at each occurrence is independently selected from the group consisting of: —O—R¹¹, —NR^{a}R^{b}, —R13, and —NR^{c}R^{d}, preferably optionally substituted C₁-C₈ alkyl and —O—R¹¹;

R¹³ at each occurrence is independently selected from the group consisting of hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; preferably optionally substituted C₁-C8 alkyl; more preferably C₁-C₈ alkyl optionally substituted with amino, hydroxy, optionally substituted phenyl, protected amino, or O(C₁-C₄ alkyl); and

R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R¹³)₂;

m is 0, 1, or 2, preferably 1;

n is 1, 2, 3, or 4, preferably 1 or 2;

X at each occurrence is independently selected from O, S, S(O), SO₂, and C(R⁷)₂, preferably CH₂or CHR⁷; provided that when m is 0, X is C(R⁷)₂; and

R⁷ at each occurrence is independently selected from the group consisting of hydrogen, halogen, cyano, —O—R¹¹, —NR^{a}R^{b}, optionally substituted aryl, optionally substituted heteroaryl, and optionally substituted —C₁-C₄ alkyl; preferably hydrogen, methyl or halogen; or two vicinal R⁷ groups can be taken together with the two adjacent atoms to which they are attached to form a fused, optionally substituted C₃-C₈ cycloalkyl or optionally substituted heterocyclic ring; preferably a fused, optionally substituted cyclopropyl; or alternatively two geminal R⁷ groups can be taken together with the carbon atom to which they are attached to form a spiro, optionally substituted C₃-C₈ cycloalkyl or optionally substituted heterocyclic ring; preferably a spiro, optionally substituted cyclopropyl.

The present invention also contemplates compounds of Formula 2-Ia, -2If, 2-Ig-1, 2-Ig-2, 2-Ig-3, 2-Ih-2Il, 2-IIa-2IId and 2-IIIa-2IIIk and pharmaceutically acceptable salts thereof as described in WO 2010/096462 wherein at least one of Y, A or Z in each of these formulae (including A¹-A⁵, Y¹-Y³ and Z¹) is substituted with -L-E or -L₃-D as defined herein. In addition, the present invention contemplates compounds of Formula 2-Iva, 2-IVb, 2-IVc, and 2-IVd and pharmaceutically acceptable salts thereof as described in WO 2010/096462 wherein the moiety between the imidazoles in each of these formulae (i.e.,

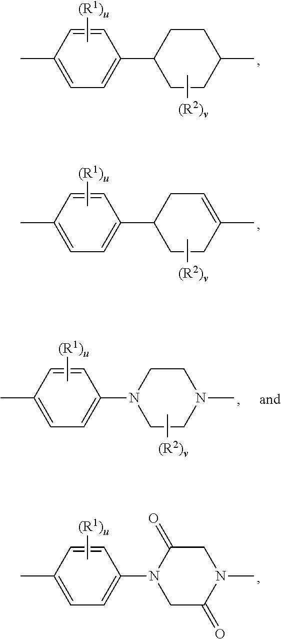

respectively) is substituted with -L-E or -L₃-D.

Except for the definitions provided for -L-E or -L₃-D provided previously herein, the remaining substitutents in the compound having above Formula VI are to be interpreted according to the meaning provided for the substitutents described in WO 2010/096462 beginning on page 65 (with the section starting “Definitions”), the contents of which are herein incorporated by reference.

Methods for making compounds of Formula VI are described in WO Publication WO2010/096462 (see compounds having the Formula 2-I) and U.S. application Ser. No. 12/959,941 filed on Dec. 3, 2010, the contents of which are herein each incorporated by reference.

In a fourth aspect, the present invention relates to compounds having the structure of below Formula (VII) or pharmaceutically acceptable salts thereof:

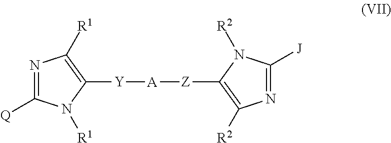

wherein:

A is (a) absent, or (b) is selected from the group consisting of an optionally substituted aliphatic group, an optionally substituted aryl, an optionally substituted heteroaryl, and a combination thereof; wherein A, if or when present, preferably is substituted with -L-E or -L₃-D as defined above in connection with the compounds of Formula II;

Y and Z at each occurrence are each independently selected from —C(O)—, —C(O)O—, —O(O)C—, —C(O)N(R^{11b})—, —N(R^{11b})C(O)—, —S(O₂)N(R^{11b})—, —N(R^{11b})S(O₂)—, —N(R^{11b})C(O)O—, —OC(O)N(R^{11b})—, and —N(R^{11b})C(O)N(R^{11c})—; wherein at least one of A, Y and Z is substituted with -L-E or -L₃-D as defined above in connection with the compounds of Formula II;

R^{11b} and R^{11e} at each occurrence is independently hydrogen or optionally substituted C₁-C₈ alkyl;

R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —CO₂R¹¹, and —C(O)NR^{a}R^{b}; preferably hydrogen, halogen and optionally substituted C₁-C₄ alkyl; wherein at least one of R¹ and R² is independently substituted with -L-E or -L₃-D as defined above in connection with the compounds of Formula II;

R¹¹ at each occurrence is independently hydrogen or optionally substituted C₁-C₈ alkyl;

R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;

Q and J are each independently selected from:

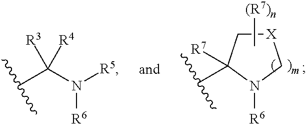

R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; preferably hydrogen or optionally substituted C₁-C₄ alkyl; or alternatively, R³ and R⁴ can be taken together with the carbon atom to which they are attached to form optionally substituted C₃-C₈ cycloalkyl or optionally substituted heterocyclic;

R⁵ at each occurrence is independently hydrogen, optionally substituted C₁-C₈ alkyl, or optionally substituted C₃-C₈ cycloalkyl; preferably hydrogen or optionally substituted C₁-C₄ alkyl;

R⁶ is selected from the group consisting of —C(O)—R¹², —C(O)—C(O)—R¹², —S(O)₂—R¹², and —C(S)—R¹²; preferably, —C(O)—R¹², more preferably an optionally substituted amino acid acyl;

R¹² at each occurrence is independently selected from the group consisting of: —O—R¹¹, NR^{a}R^{b}, —R¹³, and —NR^{c}R^{d};

R¹³ at each occurrence is independently selected from the group consisting of: hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; preferably optionally substituted C₁-C₈ alkyl; more preferably C₁-C₈ alkyl optionally substituted with amino, hydroxy, optionally substituted phenyl, protected amino, or O(C₁-C₄ alkyl); and

R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R³)₂;

m is 0, 1, or 2;

n is 1, 2, 3, or 4;

X is selected from O, S, S(O), SO₂, and C(R⁷)₂; provided that when m is 0, X is C(R⁷)₂; and

R⁷ at each occurrence is independently selected from the group consisting of: hydrogen, halogen, cyano, —O—R¹¹, —NR^{a}R^{b}, optionally substituted aryl, optionally substituted heteroaryl, and optionally substituted —C₁-C₄ alkyl; preferably hydrogen, methyl or halogen; or two vicinal R⁷ groups can be taken together with the two adjacent atoms to which they are attached to form a fused, optionally substituted C₃-C₈ cycloalkyl or optionally substituted heterocyclic ring; preferably a fused, optionally substituted cyclopropyl; or alternatively two geminal R⁷ groups can be taken together with the carbon atom to which they are attached to form a spiro, optionally substituted C₃-C₈ cycloalkyl or optionally substituted heterocyclic ring; preferably a spiro, optionally substituted cyclopropyl.

The present invention also contemplates compounds of Formula 3-Ia, -3Ii, 3-II, 3-IIa-3-IIf, 3-IIg-1, 3-IIg-2, 3-IIg-3, 3-III, 3-IIa-3-IIIf, 3-IIIg-1, 3-IIIg-2 and 3-IIIg-3 and pharmaceutically acceptable salts thereof as described in WO 2010/096462 wherein at least one of Y, A or Z in each of these formulae (including A¹-A⁹) is substituted with -L-E or -L₃-D as defined herein.

Except for the definitions provided for -L-E or -L₃-D provided previously herein, the remaining substitutents in the compound having above Formula VII are to be interpreted according to the meaning provided for the substitutents described in WO 2010/096462 beginning on page 65 (with the section starting “Definitions”), the contents of which are herein incorporated by reference.

Methods for making compounds of Formula VII (see compounds having the Formula 3-I) are described in WO2010/096462 and U.S. application Ser. No. 12/959,941 filed on Dec. 3, 2010, the contents of which are herein each incorporated by reference.

In one embodiment, the present invention features the below compounds.

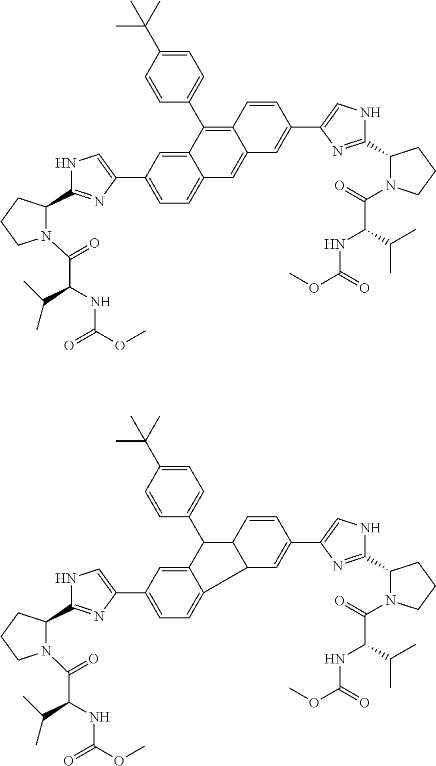 [SMILES: COC(=O)N[C@H](C(=O)N1CCC[C@H]1c1nc(-c2ccc3c(-c4ccc(C(C)(C)C)cc4)c4cc(-c5c[nH]c([C@@H]6CCCN6C(=O)[C@@H](NC(=O)OC)C(C)C)n5)ccc4cc3c2)c[nH]1)C(C)C ; COC(=O)N[C@H](C(=O)N1CCC[C@H]1c1nc(C2=CC3c4ccc(-c5c[nH]c([C@@H]6CCCN6C(=O)[C@@H](NC(=O)OC)C(C)C)n5)cc4C(c4ccc(C(C)(C)C)cc4)C3C=C2)c[nH]1)C(C)C]

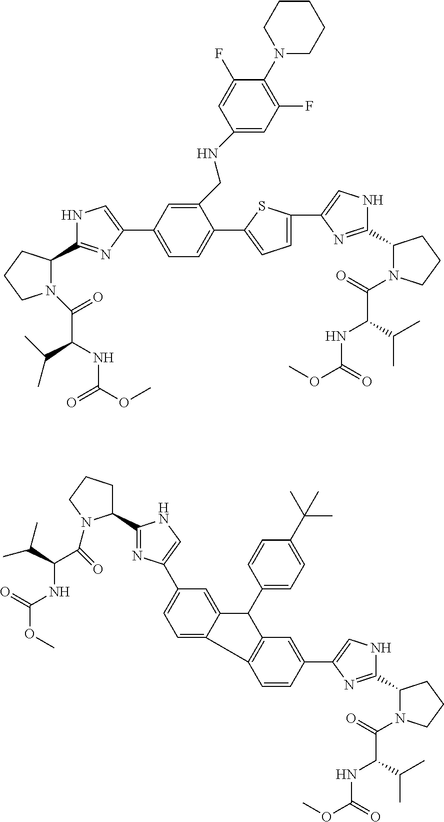 [SMILES: COC(=O)N[C@H](C(=O)N1CCC[C@H]1c1nc(-c2ccc(-c3ccc(-c4c[nH]c([C@@H]5CCCN5C(=O)[C@@H](NC(=O)OC)C(C)C)n4)s3)c(CNc3cc(F)c(N4CCCCC4)c(F)c3)c2)c[nH]1)C(C)C ; COC(=O)N[C@H](C(=O)N1CCC[C@H]1c1nc(-c2ccc3c(c2)C(c2ccc(C(C)(C)C)cc2)c2cc(-c4c[nH]c([C@@H]5CCCN5C(=O)[C@@H](NC(=O)OC)C(C)C)n4)ccc2-3)c[nH]1)C(C)C]

In another embodiment, the compounds of the invention can be prepared according to the following scheme:

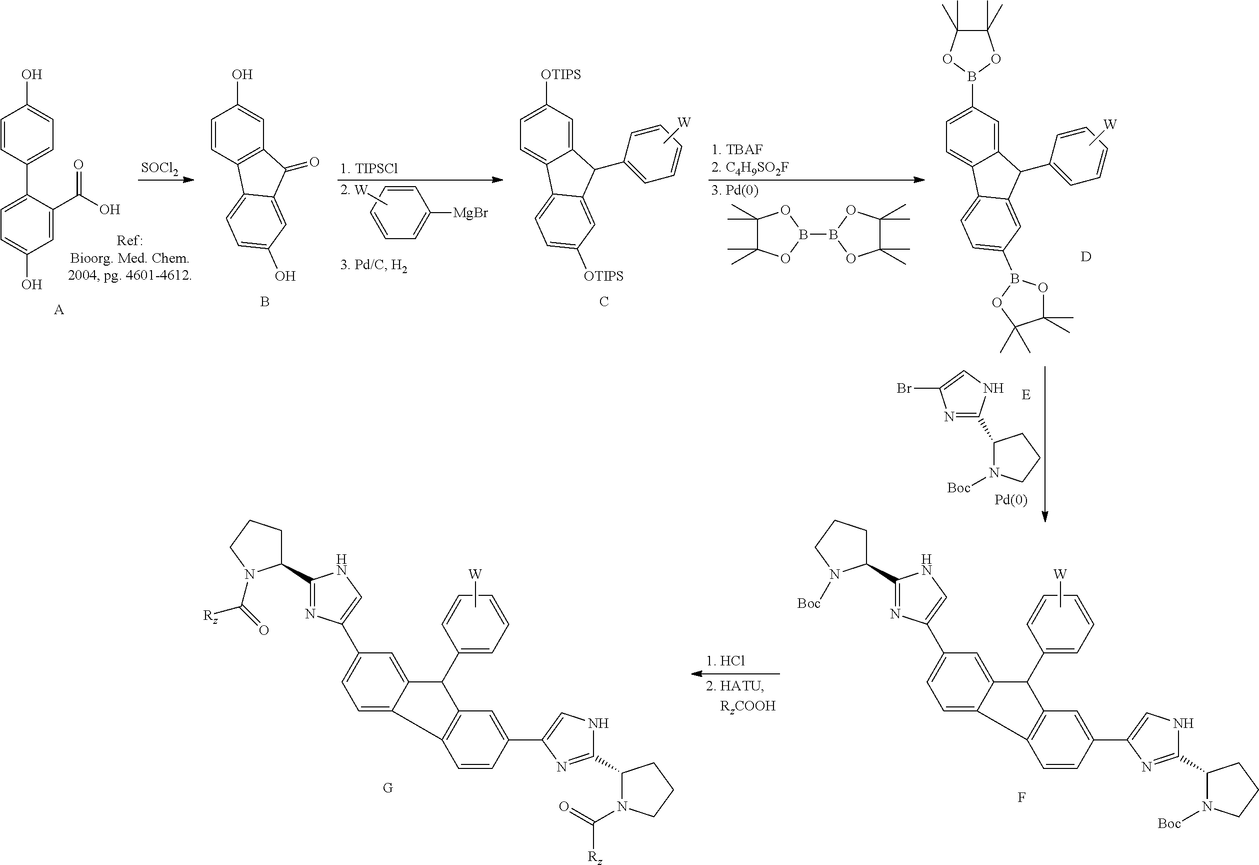

wherein R_{Z} can be, for example, R¹²; and wherein W can be, for example, hydrogen, R_{A}, or J, wherein J is C₃-C₁₂ carbocycle or 3- to 12-membered heterocycle and is optionally substituted with one or more R_{A}, or J is —SF₅.

The compounds of the present invention can be used in the form of salts. Depending on the particular compound, a salt of a compound may be advantageous due to one or more of the salt's physical properties, such as enhanced pharmaceutical stability under certain conditions or desired solubility in water or oil. In some instances, a salt of a compound may be useful for the isolation or purification of the compound.

Where a salt is intended to be administered to a patient, the salt preferably is pharmaceutically acceptable. Pharmaceutically acceptable salts include, but are not limited to, acid addition salts, base addition salts, and alkali metal salts.

Pharmaceutically acceptable acid addition salts may be prepared from inorganic or organic acids. Examples of suitable inorganic acids include, but are not limited to, hydrochloric, hydrobromic, hydroionic, nitric, carbonic, sulfuric, and phosphoric acid. Examples of suitable organic acids include, but are not limited to, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Pharmaceutically acceptable base addition salts include, but are not limited to, metallic salts and organic salts. Non-limiting examples of suitable metallic salts include alkali metal (group Ia) salts, alkaline earth metal (group IIa) salts, and other pharmaceutically acceptable metal salts. Such salts may be made, without limitation, from aluminum, calcium, lithium, magnesium, potassium, sodium, or zinc. Non-limiting examples of suitable organic salts can be made from tertiary amines and quaternary amine, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as alkyl halides (e.g., methyl, ethyl, propyl, butyl, decyl, lauryl, myristyl, and stearyl chlorides/bromides/iodides), dialkyl sulfates (e.g., dimethyl, diethyl, dibuytl, and diamyl sulfates), aralkyl halides (e.g., benzyl and phenethyl bromides), and others.

The compounds or salts of the present invention may exist in the form of solvates, such as with water (i.e., hydrates), or with organic solvents (e.g., with methanol, ethanol or acetonitrile to form, respectively, methanolate, ethanolate or acetonitrilate).

The compounds or salts of the present invention may also be used in the form of prodrugs. Some prodrugs are aliphatic or aromatic esters derived from acidic groups on the compounds of the invention. Others are aliphatic or aromatic esters of hydroxyl or amino groups on the compounds of the invention. Phosphate prodrugs of hydroxyl groups are preferred prodrugs.

The compounds of the invention may comprise asymmetrically substituted carbon atoms known as chiral centers. These compounds may exist, without limitation, as single stereoisomers (e.g., single enantiomers or single diastereomer), mixtures of stereoisomers (e.g. a mixture of enantiomers or diastereomers), or racemic mixtures. Compounds identified herein as single stereoisomers are meant to describe compounds that are present in a form that is substantially free from other stereoisomers (e.g., substantially free from other enantiomers or diastereomers). By “substantially free,” it means that at least 80% of the compound in a composition is the described stereoisomer; preferably, at least 90% of the compound in a composition is the described stereoisomer; and more preferably, at least 95%, 96%, 97%, 98% or 99% of the compound in a composition is the described stereoisomer. Where the stereochemistry of a chiral carbon is not specified in the chemical structure of a compound, the chemical structure is intended to encompass compounds containing either stereoisomer of the chiral center.

Individual stereoisomers of the compounds of this invention can be prepared using a variety of methods known in the art. These methods include, but are not limited to, stereospecific synthesis, chromatographic separation of diastereomers, chromatographic resolution of enantiomers, conversion of enantiomers in an enantiomeric mixture to diastereomers followed by chromatographically separation of the diastereomers and regeneration of the individual enantiomers, and enzymatic resolution.

Stereospecific synthesis typically involves the use of appropriate optically pure (enantiomerically pure) or substantial optically pure materials and synthetic reactions that do not cause racemization or inversion of stereochemistry at the chiral centers. Mixtures of stereoisomers of compounds, including racemic mixtures, resulting from a synthetic reaction may be separated, for example, by chromatographic techniques as appreciated by those of ordinary skill in the art. Chromatographic resolution of enantiomers can be accomplished by using chiral chromatography resins, many of which are commercially available. In a non-limiting example, racemate is placed in solution and loaded onto the column containing a chiral stationary phase. Enantiomers can then be separated by HPLC.

Resolution of enantiomers can also be accomplished by converting enantiomers in a mixture to diastereomers by reaction with chiral auxiliaries. The resulting diastereomers can be separated by column chromatography or crystallization/re-crystallization. This technique is useful when the compounds to be separated contain a carboxyl, amino or hydroxyl group that will form a salt or covalent bond with the chiral auxiliary. Non-limiting examples of suitable chiral auxiliaries include chirally pure amino acids, organic carboxylic acids or organosulfonic acids. Once the diastereomers are separated by chromatography, the individual enantiomers can be regenerated. Frequently, the chiral auxiliary can be recovered and used again.

Enzymes, such as esterases, phosphatases or lipases, can be useful for the resolution of derivatives of enantiomers in an enantiomeric mixture. For example, an ester derivative of a carboxyl group in the compounds to be separated can be treated with an enzyme which selectively hydrolyzes only one of the enantiomers in the mixture. The resulting enantiomerically pure acid can then be separated from the unhydrolyzed ester.

Alternatively, salts of enantiomers in a mixture can be prepared using any suitable method known in the art, including treatment of the carboxylic acid with a suitable optically pure base such as alkaloids or phenethylamine, followed by precipitation or crystallization/re-crystallization of the enantiomerically pure salts. Methods suitable for the resolution/separation of a mixture of stereoisomers, including racemic mixtures, can be found in ENANTIOMERS, RACEMATES, AND RESOLUTIONS (Jacques et al., 1981, John Wiley and Sons, New York, N.Y.).

A compound of this invention may possess one or more unsaturated carbon-carbon double bonds. All double bond isomers, such as the cis (Z) and trans (E) isomers, and mixtures thereof are intended to be encompassed within the scope of a recited compound unless otherwise specified. In addition, where a compound exists in various tautomeric forms, a recited compound is not limited to any one specific tautomer, but rather is intended to encompass all tautomeric forms.

Certain compounds of the invention may exist in different stable conformational forms which may be separable. Torsional asymmetry due to restricted rotations about an asymmetric single bond, for example because of steric hindrance or ring strain, may permit separation of different conformers. The invention encompasses each conformational isomer of these compounds and mixtures thereof.

Certain compounds of the invention may also exist in zwitterionic form and the invention encompasses each zwitterionic form of these compounds and mixtures thereof.

The compounds of the present invention are generally described herein using standard nomenclature. For a recited compound having asymmetric center(s), it should be understood that all of the stereoisomers of the compound and mixtures thereof are encompassed in the present invention unless otherwise specified. Non-limiting examples of stereoisomers include enantiomers, diastereomers, and cis-transisomers. Where a recited compound exists in various tautomeric forms, the compound is intended to encompass all tautomeric forms. Certain compounds are described herein using general formulas that include variables (e.g., R_{A} or R_{B}). Unless otherwise specified, each variable within such a formula is defined independently of any other variable, and any variable that occurs more than one time in a formula is defined independently at each occurrence. If moieties are described as being “independently” selected from a group, each moiety is selected independently from the other. Each moiety therefore can be identical to or different from the other moiety or moieties.

The term “pharmaceutically acceptable” is used adjectivally to mean that the modified noun is appropriate for use as a pharmaceutical product or as a part of a pharmaceutical product.

The term “therapeutically effective amount” refers to the total amount of each active substance that is sufficient to show a meaningful patient benefit, e.g. a reduction in viral load.

The term “prodrug” refers to derivatives of the compounds of the invention which have chemically or metabolically cleavable groups and become, by solvolysis or under physiological conditions, the compounds of the invention which are pharmaceutically active in vivo. A prodrug of a compound may be formed in a conventional manner by reaction of a functional group of the compound (such as an amino, hydroxy or carboxy group). Prodrugs often offer advantages of solubility, tissue compatibility, or delayed release in mammals (see, Bungard, H., DESIGN OF PRODRUGS, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Examples of prodrugs include, but are not limited to, acetate, formate, benzoate or other acylated derivatives of alcohol or amine functional groups within the compounds of the invention.

The term “solvate” refers to the physical association of a compound of this invention with one or more solvent molecules, whether organic or inorganic. This physical association often includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include, but are not limited to, hydrates, ethanolates, and methanolates.

The present invention also features pharmaceutical compositions comprising the compounds of the invention. A pharmaceutical composition of the present invention can comprise one or more compounds of the invention.

In addition, the present invention features pharmaceutical compositions comprising pharmaceutically acceptable salts, solvates, or prodrugs of the compounds of the invention. Without limitation, pharmaceutically acceptable salts can be zwitterions or derived from pharmaceutically acceptable inorganic or organic acids or bases. Preferably, a pharmaceutically acceptable salt retains the biological effectiveness of the free acid or base of the compound without undue toxicity, irritation, or allergic response, has a reasonable benefit/risk ratio, is effective for the intended use, and is not biologically or otherwise undesirable.

The present invention further features pharmaceutical compositions (a) one or more compounds of the present invention (namely, one or more of compounds having Formula (II), Formula (V), Formula (VI) or Formula (VII) or salts, solvates or prodrugs thereof; and (b) another therapeutic agent. By way of illustration not limitation, these other therapeutic agents can be selected from antiviral agents (e.g., anti-HIV agents, anti-HBV agents, or other anti-HCV agents such as HCV protease inhibitors, HCV polymerase inhibitors, HCV helicase inhibitors, IRES inhibitors or NS5A inhibitors), anti-bacterial agents, anti-fungal agents, immunomodulators, anti-cancer or chemotherapeutic agents, anti-inflammation agents, antisense RNA, siRNA, antibodies, or agents for treating cirrhosis or inflammation of the liver. Specific examples of these other therapeutic agents include, but are not limited to, ribavirin, α-interferon, β-interferon, pegylated interferon-α, pegylated interferon-lambda, ribavirin, viramidine, R-5158, nitazoxanide, amantadine, Debio-025, NIM-811, R7128, R1626, R4048, T-1106, PSI-7851, PF-00868554, ANA-598, IDX184, IDX102, IDX375, GS-9190, VCH-759, VCH-916, MK-3281, BCX-4678, MK-3281, VBY708, ANA598, GL59728, GL60667, BMS-790052, BMS-791325, BMS-650032, GS-9132, ACH-1095, AP-H005, A-831, A-689, AZD2836, telaprevir, boceprevir, ITMN-191, BI-201335, VBY-376, VX-500 (Vertex), PHX-B, ACH-1625, IDX136, IDX316, VX-813 (Vertex), SCH 900518 (Schering-Plough), TMC-435 (Tibotec), ITMN-191 (Intermune, Roche), MK-7009 (Merck), IDX-PI (Novartis), BI-201335 (Boehringer Ingelheim); R7128 (Roche), PSI-7851 (Pharmasset), MK-3281 (Merck), PF-868554 (Pfizer), IDX-184 (Novartis), IDX-375 (Pharmasset), BILB-1941 (Boehringer Ingelheim), GS-9190 (Gilead), BMS-790052 (BMS), ABT-450 (Abbott/Enanta), ABT-072 (Abbott), ABT-333 (Abbott), Albuferon (Novartis), ritonavir, another cytochrome P450 monooxygenase inhibitor, or any combination thereof.

In one embodiment, a pharmaceutical composition of the present invention comprises (a) one or more compounds of the present invention (namely, one or more of compounds having Formula (II), Formula (V), Formula (VI) or Formula (VII) or salts, solvates or prodrugs thereof; and (b) one or more other antiviral agents.

In another embodiment, a pharmaceutical composition of the present invention comprises (a) one or more compounds of the present invention (namely, one or more of compounds having Formula (II), Formula (V), Formula (VI) or Formula (VII) or salts, solvates or prodrugs thereof; and (b) and one or more other anti-HCV agents, such as an agent selected from HCV polymerase inhibitors (including nucleoside or non-nucleoside type of polymerase inhibitors), HCV protease inhibitors, HCV helicase inhibitors, CD81 inhibitors, cyclophilin inhibitors, IRES inhibitors, or NS5A inhibitors.

In yet another embodiment, a pharmaceutical composition of the present invention comprises (a) one or more compounds of the present invention (namely, one or more of compounds having Formula (II), Formula (V), Formula (VI) or Formula (VII) or salts, solvates or prodrugs thereof; and (b) one or more other antiviral agents, such as anti-HBV, anti-HIV agents, or anti-hepatitis A, anti-hepatitis D, anti-hepatitis E or anti-hepatitis G agents. Non-limiting examples of anti-HBV agents include adefovir, lamivudine, and tenofovir. Non-limiting examples of anti-HIV drugs include ritonavir, lopinavir, indinavir, nelfinavir, saquinavir, amprenavir, atazanavir, tipranavir, TMC-114, fosamprenavir, zidovudine, lamivudine, didanosine, stavudine, tenofovir, zalcitabine, abacavir, efavirenz, nevirapine, delavirdine, TMC-125, L-870812, S-1360, enfuvirtide, T-1249, or other HIV protease, reverse transcriptase, integrase or fusion inhibitors. Any other desirable antiviral agents can also be included in a pharmaceutical composition of the present invention, as appreciated by those skilled in the art.

A pharmaceutical composition of the present invention typically includes a pharmaceutically acceptable carrier or excipient. Non-limiting examples of suitable pharmaceutically acceptable carriers/excipients include sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, or phosphate buffer solutions. Lubricants, coloring agents, releasing agents, coating agents, sweetening, flavoring or perfuming agents, preservatives, or antioxidants can also be included in a pharmaceutical composition of the present invention.

The pharmaceutical compositions of the present invention can be formulated based on their routes of administration using methods well known in the art. For example, a sterile injectable preparation can be prepared as a sterile injectable aqueous or oleagenous suspension using suitable dispersing or wetting agents and suspending agents. Suppositories for rectal administration can be prepared by mixing drugs with a suitable nonirritating excipient such as cocoa butter or polyethylene glycols which are solid at ordinary temperatures but liquid at the rectal temperature and will therefore melt in the rectum and release the drugs. Solid dosage forms for oral administration can be capsules, tablets, pills, powders or granules. In such solid dosage forms, the active compounds can be admixed with at least one inert diluent such as sucrose lactose or starch. Solid dosage forms may also comprise other substances in addition to inert diluents, such as lubricating agents. In the case of capsules, tablets and pills, the dosage forms may also comprise buffering agents. Tablets and pills can additionally be prepared with enteric coatings. Liquid dosage forms for oral administration can include pharmaceutically acceptable emulsions, solutions, suspensions, syrups or elixirs containing inert diluents commonly used in the art. Liquid dosage forms may also comprise wetting, emulsifying, suspending, sweetening, flavoring, or perfuming agents. The pharmaceutical compositions of the present invention can also be administered in the form of liposomes, as described in U.S. Pat. No. 6,703,403. Formulation of drugs that are applicable to the present invention is generally discussed in, for example, Hoover, John E., REMINGTON'S PHARMACEUTICAL SCIENCES (Mack Publishing Co., Easton, Pa.: 1975), and Lachman, L., eds., PHARMACEUTICAL DOSAGE FORMS (Marcel Decker, New York, N.Y., 1980).

Any compound described herein (i.e, any compounds having a Formula (II), Formula (V), Formula (VI) or Formula (VII) or a pharmaceutically acceptable salt thereof, can be used to prepared pharmaceutical compositions of the present invention.

The present invention further features methods of using the compounds of the present (namely, one or more of compounds having Formula (II), Formula (V), Formula (VI) or Formula (VII) or salts, solvates or prodrugs thereof to inhibit HCV replication. The methods comprise contacting cells infected with HCV virus with an effective amount of a compound of the present invention (namely, one or more of compounds having Formula (II), Formula (V), Formula (VI) or Formula (VII) or salts, solvates or prodrugs thereof thereby inhibiting the replication of HCV virus in the cells. As used herein, “inhibiting” means significantly reducing, or abolishing, the activity being inhibited (e.g., viral replication). In many cases, representative compounds of the present invention can reduce the replication of HCV virus (e.g., in an HCV replicon assay as described above) by at least 10%, 20%, 30%, 40%, 50%, 60%, 70%, 80%, 90%, 95% or more.

The compounds of the present invention may inhibit one or more HCV subtypes. Examples of HCV subtypes that are amenable to the present invention include, but are not be limited to, HCV genotypes 1, 2, 3, 4, 5 and 6, including HCV genotypes 1a, 1b, 2a, 2b, 2c or 3a. In one embodiment, a compound or compounds of the present invention (or salts, solvates or prodrugs thereof) are used to inhibit the replication of HCV genotype 1a. In another embodiment, a compound or compounds of the present invention (or salts, solvates or prodrugs thereof) are used to inhibit the replication of HCV genotype 1b. In still another embodiment, a compound or compounds of the present invention (or salts, solvates or prodrugs thereof) are used to inhibit the replication of both HCV genotypes 1a and 1b.

The present invention also features methods of using the compounds of the present invention (or salts, solvates or prodrugs thereof) to treat HCV infection. The methods typically comprise administering a therapeutic effective amount of a compound of the present invention (or a salt, solvate or prodrug thereof), or a pharmaceutical composition comprising the same, to an HCV patient, thereby reducing the HCV viral level in the blood or liver of the patient. As used herein, the term “treating” refers to reversing, alleviating, inhibiting the progress of, or preventing the disorder or condition, or one or more symptoms of such disorder or condition to which such term applies. The term “treatment” refers to the act of treating. In one embodiment, the methods comprise administering a therapeutic effective amount of two or more compounds of the present invention (or salts, solvates or prodrugs thereof), or a pharmaceutical composition comprising the same, to an HCV patient, thereby reducing the HCV viral level in the blood or liver of the patient.

A compound of the present invention (or a salt, solvate or prodrug thereof) can be administered as the sole active pharmaceutical agent, or in combination with another desired drug, such as other anti-HCV agents, anti-HIV agents, anti-HBV agents, anti-hepatitis A agents, anti-hepatitis D agents, anti-hepatitis E agents, anti-hepatitis G agents, or other antiviral drugs. Any compound described herein, or a pharmaceutically acceptable salt thereof, can be employed in the methods of the present invention.

A compound of the present invention (namely, one or more of compounds having Formula (II), Formula (V), Formula (VI) or Formula (VII) or salts, solvates or prodrugs thereof can be administered to a patient in a single dose or divided doses. A typical daily dosage can range, without limitation, from 0.1 to 200 mg/kg body weight, such as from 0.25 to 100 mg/kg body weight. Single dose compositions can contain these amounts or submultiples thereof to make up the daily dose. Preferably, each dosage contains a sufficient amount of a compound of the present invention that is effective in reducing the HCV viral load in the blood or liver of the patient. The amount of the active ingredient, or the active ingredients that are combined, to produce a single dosage form may vary depending upon the host treated and the particular mode of administration. It will be understood that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy.

The present invention further features methods of using the pharmaceutical compositions of the present invention to treat HCV infection. The methods typically comprise administering a pharmaceutical composition of the present invention to an HCV patient, thereby reducing the HCV viral level in the blood or liver of the patient. Any pharmaceutical composition described herein can be used in the methods of the present invention.

In addition, the present invention features use of the compounds or salts of the present invention for the manufacture of medicaments for the treatment of HCV infection. Any compound described herein, or a pharmaceutically acceptable salt thereof, can be used to make medicaments of the present invention.

The compounds of the present invention can also be isotopically substituted. Preferred isotopic substitution include substitutions with stable or nonradioactive isotopes such as deuterium, ¹³C, ¹⁵N or ¹⁸O. Incorporation of a heavy atom, such as substitution of deuterium for hydrogen, can give rise to an isotope effect that could alter the pharmacokinetics of the drug. In one example, at least 10 mol % of hydrogen in a compound of the present invention is substituted with deuterium. In another example, at least 25 mole % of hydrogen in a compound of the present invention is substituted with deuterium. In a further example, at least 50, 60, 70, 80 or 90 mole % of hydrogen in a compound of the present invention is substituted with deuterium. The natural abundance of deuterium is about 0.015%. Deuterium substitution or enrichment can be achieved, without limitation, by either exchanging protons with deuterium or by synthesizing the molecule with enriched or substituted starting materials. Other methods known in the art can also be used for isotopic substitutions.

The compounds of the present invention can also be isotopically substituted. Preferred isotopic substitution include substitutions with stable or nonradioactive isotopes such as deuterium, ¹³C, ¹⁵N or ¹⁸O. Incorporation of a heavy atom, such as substitution of deuterium for hydrogen, can give rise to an isotope effect that could alter the pharmacokinetics of the drug. In one example, at least 10 mol % of hydrogen in a compound of the present invention is substituted with deuterium. In another example, at least 25 mole % of hydrogen in a compound of the present invention is substituted with deuterium. In a further example, at least 50, 60, 70, 80 or 90 mole % of hydrogen in a compound of the present invention is substituted with deuterium. The natural abundance of deuterium is about 0.015%. Deuterium substitution or enrichment can be achieved, without limitation, by either exchanging protons with deuterium or by synthesizing the molecule with enriched or substituted starting materials. Other methods known in the art can also be used for isotopic substitutions.

The contents of all references (including literature references, issued patents, published patent applications, and co-pending patent applications) cited throughout this application are hereby expressly incorporated herein in their entireties by reference.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A compound of Formula (II) or pharmaceutically acceptable salts thereof: 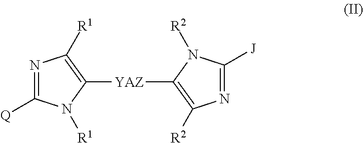
wherein:
A is (a) absent; or (b) a cyclic group independently selected from aryl, heteroaryl, heterocyclic, C₃-C₈ cycloalkyl, and C₃-C₈ cycloalkenyl, each optionally substituted; wherein A, when present, is substituted with -L-E or -L₃-D, wherein L, E, L₃ and D are defined as below;
Y is (a) absent; or (b) an optionally substituted aliphatic group selected from the group consisting of O, S, N(R¹¹), C₁-C₈, alkyl, C₂-C₈, alkenyl, C₂-C₈, alkynyl, C₃-C₈, cycloalkyl, C₃-C₈ cycloalkenyl, and heterocyclic, each optionally substituted; or a combination thereof; wherein Y, when present, is substituted with -L-E or -L₃-D, wherein L, E, L₃ and D are defined as below;
Z is (a) absent; or (b) an optionally substituted linear aliphatic group; optionally Z contains a group selected from O, N(R¹¹), C(O), S(O)₂, C(O)O, C(O)N(R¹¹), OC(O)O, OC(O)N(R¹¹), S(O)₂N(R¹¹), N(R¹¹)C(O)N(R¹¹), N(R¹¹)C(O)C(O)N(R¹¹), N(R¹¹)S(O)₂N(R¹¹), C(O)N(R¹¹)S(O)₂ and C(O)N(R¹¹)S(O)₂N(R¹¹); wherein Z, when present, is substituted with -L-E or -L₃-D, wherein L, E, L₃ and D are defined as below;
wherein at least two of A, Y, and Z are optionally present;
R¹¹ at each occurrence is independently hydrogen or optionally substituted C₁-C₈ alkyl;
R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —CO₂R¹¹, and —C(O)NR^{a}R^{b}; wherein at least one of R¹ and R² independently substituted with -L-E or -L₃-D as defined below;
R^{a} and R^{b} at each occurrence are each independently selected from the group consisting of hydrogen, optionally substituted C₁-C₈, alkyl, and optionally substituted C₂-C₈, alkenyl; or R^{a} and R^{b} can be taken together with the nitrogen atom to which they are attached to form an optionally substituted heterocyclic or optionally substituted heteroaryl group;
Q and J are each independently selected from: 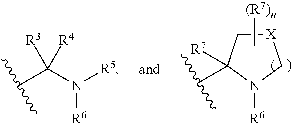
R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈, cycloalkyl or optionally substituted heterocyclic;
R⁵ at each occurrence is independently hydrogen, optionally substituted C₁-C₈, alkyl, or optionally substituted C₃-C₈ cycloalkyl;
R⁶ at each occurrence is independently selected from the group consisting of —C(O)—R¹², —C(O)—C(O)—R¹², —S(O)₂—R¹², and —C(S)—R¹²;
R¹² at each occurrence is independently selected from the group consisting of: optionally substituted C₁-C₈ alkyl, —O—R¹¹, —NR^{a}R^{b}, —R¹³, and —NR^{c}R^{d};
R¹³ at each occurrence is independently selected from the group consisting of hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and
R^{c} and R^{d} at each occurrence are each independently selected from the group consisting of hydrogen, —R³, —C(O)—R¹³, —C(O)—OR¹³, —S(O)₂—R¹³, —C(O)N(R¹³)₂, and —S(O)₂N(R¹³)₈;
m is 0, 1, or 2;
n is 1, 2, 3, or 4;
X at each occurrence is independently selected from O, S, S(O), SO₂, and C(R⁷)₂ provided that when m is 0, X is C(R⁷)₂; and
R⁷ at each occurrence is independently selected from the group consisting of hydrogen, halogen, —C₁-C₄ alkyl, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; optionally substituted cyclopropyl; or alternatively two geminal R⁷ groups can be taken together with the carbon atom to which they are attached to form a spiro, optionally substituted C₃-C₈ cycloalkyl or optionally substituted heterocyclic ring,
-L-E or -L₃-D are as follows:
E is (i) C₃-C₁₄ carbocycle or 3- to 14-membered heterocycle, and is optionally substituted with one or more R_{A}; or (ii) E is -L_{S}-R_{E};
L is -L_{S}-, -L_{S}-O-L_{S}′, -L_{S}-C(O)-L_{S}′-, -L_{S}-S(O)₂-L_{S}′, -L_{S}-S(O)-L_{S}′-, -L_{S}-OS(O)₂-L_{S}′-, -L_{S}-S(O)₂O-L_{S}′-, -L_{S}-OS(O)-L_{S}′-, -L_{S}-S(O)O-L_{S}′-, -L_{S}-C(O)O-L_{S}′-, -L_{S}-OC(O)-L_{S}′-, -L_{S}-OC(O)O-L_{S}′-, -L_{S}-C(O)N(R_{B})-L_{S}′-, -L_{S}-N(R_{B})C(O)-L_{S}′-, -L_{S}-C(O)N(R_{B})O-L_{S}′-, -L_{S}-N(R_{B})C(O)O-L_{S}′-, -L_{S}-OC(O)N(R_{B})-L_{S}′-, -L_{S}-C(O)N(R)N(R_{B})-L_{S}′-, -L_{S}-S-L_{S}′-, -L_{S}-C(S)-L_{S}′-, -L_{S}-C(S)O-L_{S}′-, -L_{S}-OC(S)-L_{S}′-, -L_{S}-C(S)N(R_{B})-L_{S}′-, -L_{S}-N(R_{B})-L_{S}′-, -L_{S}-N(R_{B})C(S)-L_{S}′-, -L_{S}-N(R)S(O)-L_{S}′-, -L_{S}-N(R_{B})S(O)₂-L_{S}′-, -L_{S}-S(O)₂N(R_{B})-L_{S}′-, -L_{S}-S(O)N(R_{B})-L_{S}′-, -L_{S}-C(S)N(R_{B})O-L_{S}′-, -L_{S}-C(O)N(R_{B})C(O)-L_{S}′-, -L_{S}-N(R_{B})C(O)N(R_{B}′)-L_{S}′-, -L_{S}-N(R_{B})SO₂N(R_{B}′)-L_{S}′-, -L_{S}-N(R)S(O)N(R_{B}′)-L_{S}′-, or -L_{S}-C(S)N(R_{B})N(R_{B}′)-L_{S}′-;
L_{S} and L_{S}′ are each independently selected at each occurrence from bond; or C₁-C₆ alkylene, C₂-C₆ alkenylene or C₂-C₆ alkynylene, each of which is independently optionally substituted at each occurrence with one or more R_{L};
R_{A} is independently selected at each occurrence from halogen, oxo, thioxo, hydroxy, mercapto, nitro, cyano, amino, carboxy, formyl, phosphonoxy, or phosphono; or -L_{S}-R_{E};
R_{B} and R_{B′} are each independently selected at each occurrence from hydrogen; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, C₃-C₆ carbocycle or 3- to 6-membered heterocycle; or C₃-C₆ carbocycle or 3- to 6-membered heterocycle; wherein each C₃-C₆ carbocycle or 3- to 6-membered heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆haloalkenyl or C₂-C₆ haloalkynyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R_{S}″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′) or —C(O)N(R_{S})C(O)—R_{S}′; or C₁-C₆ alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆ carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl;
R_{S}, R_{S}′ and R_{S}″ are each independently selected at each occurrence from hydrogen; C₁-C₆ alkyl, C₂-C₆alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆haloalkynyl;
L₃ is bond or -L_{S}-K-L_{S}′-, wherein K is selected from bond, —O—, —S—, —N(R_{B})—, —C(O)—, —S(O)₂—, —S(O)—, —OS(O)—, —OS(O)₂—, —S(O)₂O—, —S(O)O—, —C(O)O—, —OC(O)—, —OC(O)O—, —C(O)N(R_{B})—, —N(R_{B})C(O)—, —N(R_{B})C(O)O—, —OC(O)N(R_{B})—, —N(R_{B})S(O)—, —N(R_{B})S(O)₂—, —S(O)N(R_{B})—, —S(O)₂N(R_{B})—, —C(O)N(R_{B})C(O)—, —N(R_{B})C(O)N(R_{B}′)—, —N(R_{B})SO₂N(R_{B}′)—, or —N(R_{B})S(O)N(R_{B}′)—;
D is C₃-C₁₂ carbocycle or 3- to 12-membered heterocycle, and is optionally substituted with one or more R_{A}; or D is C₃-C₁₂ carbocycle or 3- to 12-membered heterocycle which is substituted with J and optionally substituted with one or more R_{A}, where J is C₃-C₁₂ carbocycle or 3- to 12-membered heterocycle and is optionally substituted with one or more R_{A}, or J is —SF₅; or D is hydrogen or R_{A};
R_{A} is independently selected at each occurrence from halogen, nitro, oxo, phosphonoxy, phosphono, thioxo, cyano, or -L_{S}-R_{E}, wherein two adjacent R_{A}, taken together with the atoms to which they are attached and any atoms between the atoms to which they are attached, can optionally form carbocycle or heterocycle;
R_{B} and R_{B}′ are each independently selected at each occurrence from hydrogen; or C₁-C₆alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano or 3- to 6-membered carbocycle or heterocycle; or 3- to 6-membered carbocycle or heterocycle; wherein each 3- to 6-membered carbocycle or heterocycle in R_{B} or R_{B}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆haloalkenyl or C₂-C₆haloalkynyl;
R_{E} is independently selected at each occurrence from —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′), —N(R_{S})C(O)R_{S}′, —N(R_{S})C(O)N(R_{S}′R₅″), —N(R_{S})SO₂R_{S}′, —SO₂N(R_{S}R_{S}′), —N(R_{S})SO₂N(R_{S}′R_{S}″), —N(R_{S})S(O)N(R_{S}′R_{S}″), —OS(O)—R_{S}, —OS(O)₂—R_{S}, —S(O)₂OR_{S}, —S(O)OR_{S}, —OC(O)OR_{S}, —N(R_{S})C(O)OR_{S}′, —OC(O)N(R_{S}R_{S}′), —N(R_{S})S(O)—R_{S}′, —S(O)N(R_{S}R_{S}′), —P(O)(OR_{S})₂, or —C(O)N(R_{S})C(O)—R_{S}′; or C₁-C₆alkyl, C₂-C₆ alkenyl or C₂-C₆alkynyl, each of which is independently optionally substituted at each occurrence with one or more substituents selected from halogen, hydroxy, mercapto, amino, carboxy, nitro, oxo, phosphonoxy, phosphono, thioxo, formyl or cyano; or C₃-C₆ carbocycle or 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆alkenyl, C₂-C₆ alkynyl, C₁-C₆ haloalkyl, C₂-C₆ haloalkenyl, C₂-C₆ haloalkynyl, C(O)OR_{S}, or —N(R_{S}R_{S}′);
R_{L} is independently selected at each occurrence from halogen, nitro, oxo, phosphonoxy, phosphono, thioxo, cyano, —O—R_{S}, —S—R_{S}, —C(O)R_{S}, —OC(O)R_{S}, —C(O)OR_{S}, —N(R_{S}R_{S}′), —S(O)R_{S}, —SO₂R_{S}, —C(O)N(R_{S}R_{S}′) or —N(R_{S})C(O)R_{S}′; or C₃-C₆ carbocycle 3- to 6-membered heterocycle, each of which is independently optionally substituted at each occurrence with one or more substituents selected from halogen, hydroxy, mercapto, amino, carboxy, nitro, oxo, phosphonoxy, phosphono, thioxo, formyl, cyano, C₁-C₆alkyl, C₂-C₆alkenyl, C₂-C₆alkynyl, C₁-C₆haloalkyl, C₂-C₆ haloalkenyl or C₂-C₆ haloalkynyl; wherein two adjacent R_{L}, taken together with the atoms to which they are attached and any atoms between the atoms to which they are attached, can optionally form carbocycle or heterocycle;
L_{S} and L_{S}′ are each independently selected at each occurrence from bond; or C₁-C₆alkylene, C₂-C₆alkenylene or C₂-C₆alkynylene, each of which is independently optionally substituted at each occurrence with one or more R_{L}; and
R_{S}, R_{S}′ and R_{S}″ are each independently selected at each occurrence from hydrogen; C₁-C₆alkyl, C₂-C₆ alkenyl or C₂-C₆ alkynyl, each of which is independently optionally substituted at each occurrence with one or more substituents selected from halogen, hydroxy, mercapto, amino, carboxy, nitro, oxo, phosphonoxy, phosphono, thioxo, formyl, cyano, —O—C₁-C₆ alkyl, —O—C₁-C₆ alkylene-O—C₁-C₆ alkyl, or 3- to 6-membered carbocycle or heterocycle; or 3- to 6-membered carbocycle or heterocycle; wherein each 3- to 6-membered carbocycle or heterocycle in R_{S}, R_{S}′ or R_{S}′ is independently optionally substituted at each occurrence with one or more substituents selected from halogen, hydroxy, mercapto, amino, carboxy, nitro, oxo, phosphonoxy, phosphono, thioxo, formyl, cyano, C₁-C₆ alkyl, C₂-C₆ alkenyl, C₂-C₆ alkynyl, C₁-C₆ haloalkyl, C₂-C₆ haloalkenyl or C₂-C₆ haloalkynyl.

2. A compound of Formula (V) or pharmaceutically acceptable salts thereof: 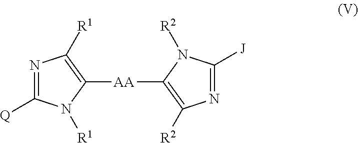
wherein:
AA is an aromatic group selected from the group consisting of:
i. a bicyclic aryl or bicyclic heteroaryl, wherein the rings of said bicyclic ring system are fused or covalently attached, wherein each imidazole moiety is attached to a different ring of said bicyclic ring system and wherein at least one ring of said bicyclic is a 5- or 7-membered ring;
ii. a tricyclic aryl or tricyclic heteroaryl, wherein the rings of said tricyclic ring system are fused, covalently attached or a combination thereof, and wherein each imidazole moiety is attached to a different ring of said tricyclic; and 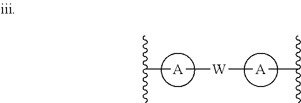
each A is independently a 5-7 membered aryl or heteroaryl, wherein said aryl or heteroaryl are optionally substituted; or
wherein AA is substituted with -L-E or -L₃-D, wherein L, E, L₃ and D are defined as in claim 1;
W is an aliphatic group containing zero to eight carbons;
R¹¹ at each occurrence is independently hydrogen or optionally substituted C₁-C₈ alkyl;
R¹ and R² at each occurrence are each independently selected from the group consisting of: halogen, cyano, optionally substituted C₁-C₄ alkyl, —O—R¹¹, —NR^{a}R^{b}, —C(O)R¹¹, —CO₂R¹¹, and —C(O)NR^{a}R^{b}; wherein at least one of R¹ and R² can be substituted with -L-E or -L₃-D wherein L, E, L₃ and D are defined as in claim 1;
R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;
Q and J are each independently selected from: 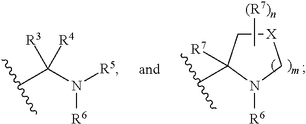
R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈ cycloalkyl or optionally substituted heterocyclic;
R⁵ at each occurrence is independently hydrogen, optionally substituted C₁-C₈ alkyl, or optionally substituted C₃-C₈ cycloalkyl;
R⁶ at each occurrence is independently, selected from the group consisting of —C(O)—R¹², —C(O)—C(O)—R¹², —S(O)₂—R¹², and —C(S)—R¹²;
R¹² at each occurrence is independently selected from the group consisting of: —O—R¹¹, —NR^{a}—R^{b}, —R¹³, —NR^{c}R^{d};
R¹³ at each occurrence is independently selected from the group consisting of hydrogen, C₁-C₈ alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and
R^{c} and R^{d} at each occurrence are each independently selected from the group consisting of hydrogen, —R¹³, —C(O)—R³, —C(O)—OR³, —S(O)₂—R¹³, —C(O)N(R³)₂, and —S(O)₂N(R¹³)₂;
m is 0, 1, or 2;
n is 1, 2, 3, or 4;
X at each occurrence is independently selected from O, S, S(O), SO₂, and C(R⁷)₂; provided that when m is 0, X is C(R⁷)₂; and
R⁷ at each occurrence is independently selected from the group consisting of hydrogen, halogen, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; or alternatively two geminal R⁷ groups can be taken together with the carbon atom to which they are attached to form a spiro, optionally substituted C₃-C₈ cycloalkyl or optionally substituted heterocyclic ring.

3. A compound of Formula (VI) or pharmaceutically acceptable salts thereof: 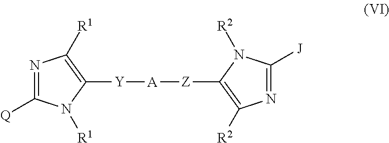
A is (a) absent; or (b) a cyclic group independently selected from aryl, heteroaryl, heterocyclic, C₃-C₈ cycloalkyl, and C₃-C₈ cycloalkenyl, each optionally substituted; wherein A, when is present, is substituted with -L-E or -L₃-D, wherein L, E, L₃ and D are defined in claim 1;
Y is (a) absent; or (b) an optionally substituted aliphatic group selected from the group consisting of O, S, N(R¹¹), C₁-C₈ alkyl, C₂-C₈ alkenyl, C₂-C₈ alkynyl, C₃-C₈ cycloalkyl, C₃-C₈ cycloalkenyl, and heterocyclic, each optionally substituted; or a combination thereof; wherein Y, when present, is substituted with -L-E or -L₃-D, wherein L, E, L₃ and D are defined above in claim 1;
Z is (a) absent; or (b) an optionally substituted linear aliphatic group; optionally Z contains a group selected from O, N(R¹¹), C(O), S(O)₂, C(O)O, C(O)N(R¹¹), OC(O)O, OC(O)N(R¹¹), S(O)₂N(R¹¹), N(R¹¹)C(O)N(R¹¹), N(R¹¹)C(O)C(O)N(R¹¹), N(R¹¹)S(O)₂N(R¹¹), C(O)N(R¹¹)S(O)₂ and C(O)N(R¹¹)S(O)₂N(R¹¹); wherein Z, when present, is substituted with -L-E or -L₃-D, wherein L, E, L₃ and D are defined in claim 1;
wherein at least two of A, Y, and Z are present;
R¹¹ at each occurrence is independently hydrogen or optionally substituted C₁-C₈ alkyl;
R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —C(O)₂R¹¹, and —C(O)NR^{a}R^{b}; wherein at least one of R¹ and R² can be substituted with -L-E or -L₃-D wherein L, E, L₃ and D are defined as in claim 1;
R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;
Q and J are each independently selected from: 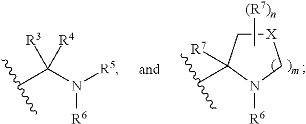
R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈ cycloalkyl or optionally substituted heterocyclic;
R⁵ at each occurrence is independently hydrogen, optionally substituted C₁-C₈ alkyl, or optionally substituted C₃-C₈ cycloalkyl;
R⁶ at each occurrence is independently selected from the group consisting of —C(O)—R¹², —C(O)—C(O)—R¹², —S(O)₂—R¹², and —C(S)—R¹²;
R¹² at each occurrence is independently selected from the group consisting of: —O—R¹¹, —NR^{a}R^{b}, —R13, and —NR^{c}R^{d};
R¹³ at each occurrence is independently selected from the group consisting of hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and
R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R¹³)₂;
m is 0, 1, or 2;
n is 1, 2, 3, or 4;
X at each occurrence is independently selected from O, S, S(O), SO₂, and C(R⁷)₂; provided that when m is 0, X is C(R⁷)₂; and
R⁷ at each occurrence is independently selected from the group consisting of hydrogen, halogen, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; or alternatively two geminal R⁷ groups can be taken together with the carbon atom to which they are attached to form a spiro, optionally substituted C₃-C₈ cycloalkyl or optionally substituted heterocyclic ring.

4. A compound of Formula (VII) or pharmaceutically acceptable salts thereof: 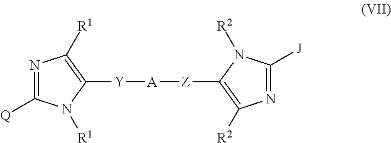
wherein:
A is (a) absent, or (b) is selected from the group consisting of an optionally substituted aliphatic group, an optionally substituted aryl, an optionally substituted heteroaryl, and a combination thereof; wherein A, when present, is substituted with -L-E or -L₃-D, wherein L, E, L₃ and D are defined in claim 1;
Y and Z at each occurrence are each independently selected from —C(O)—, —C(O)O—, —O(O)C—, —C(O)N(R^{11b})—, —N(R^{11b})C(O)—, —S(O₂)N(R^{11b})—, —N(R^{11b})S(O₂)—, —N(R^{11b})C(O)O—, —OC(O)N(R^{11b})—, and —N(R^{11b})C(O)N(R^{11c})—; wherein Y or Z, when present, is substituted with -L-E or -L₃-D, wherein L, E, L₃ and D are defined in claim 1;
R^{11b} and R^{11c} at each occurrence is independently hydrogen or optionally substituted C₁-C₈ alkyl;
R¹ and R² at each occurrence are each independently selected from the group consisting of hydrogen, halogen, cyano, optionally substituted C₁-C₄ alkyl, —O—R¹¹, —NR^{a}R^{b}, —C(O)R¹¹, —CO₂R¹¹, and —C(O)NR^{a}R^{b}; wherein at least one of R¹ and R² can be substituted with -L-E or -L₃-D wherein L, E, L₃ and D are defined as in claim 1;
R¹¹ at each occurrence is independently hydrogen or optionally substituted C₁-C₈ alkyl;
R^{a} and R^{b} at each occurrence are each independently selected from the group consisting of hydrogen, optionally substituted C₁-C₈ alkyl, and optionally substituted C₂-C₈ alkenyl; or R^{a} and R^{b} can be taken together with the nitrogen atom to which they are attached to form an optionally substituted heterocyclic or optionally substituted heteroaryl group;
Q and J are each independently selected from: 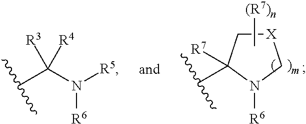
R³ and R⁴ at each occurrence are each independently selected from the group consisting of hydrogen, optionally substituted C₁-C₈ alkyl, optionally substituted C₂-C₈ alkenyl, and optionally substituted C₃-C₈ cycloalkyl; or alternatively, R³ and R⁴ can be taken together with the carbon atom to which they are attached to form optionally substituted C₃-C₈ cycloalkyl or optionally substituted heterocyclic;
R⁵ at each occurrence is independently hydrogen, optionally substituted C₁-C₈ alkyl, or optionally substituted C₃-C₈ cycloalkyl;
R⁶ is selected from the group consisting of —C(O)—R¹², —C(O)—C(O)—R¹², —S(O)₂—R¹², and —C(S)—R¹²;
R¹² at each occurrence is independently selected from the group consisting of: —O—R¹¹, NR^{a}R^{b}, —R¹³, and —NR^{c}R^{d};
R¹¹ at each occurrence is independently selected from the group consisting of: hydrogen, C₁-C₈alkyl, C₂-C₈ alkenyl, C₂-C₈ alkynyl, C₃-C₈ cycloalkyl, C₃-C₈ cycloalkenyl, heterocyclic, aryl, and heteroaryl, each optionally substituted; and
R^{c} and R^{d} at each occurrence are each independently selected from the group consisting of hydrogen, —R¹³, —C(O)—R¹³, —C(O)—OR¹³, —S(O)₂—R¹³, —C(O)N(R¹³)₂, and —S(O)₂N(R¹³)₂;
m is 0, 1, or 2;
n is 1, 2, 3, or 4;
X is selected from O, S, S(O), SO₂, and C(R⁷)₂; provided that when m is 0, X is C(R⁷)₂; and
R⁷ at each occurrence is independently selected from the group consisting of: hydrogen, halogen, cyano, —O—R¹¹, —NR^{a}R^{b}, optionally substituted aryl, optionally substituted heteroaryl, and optionally substituted —C₁-C₄ alkyl; or two vicinal R⁷ groups can be taken together with the two adjacent atoms to which they are attached to form a fused, optionally substituted C₃-C₈ cycloalkyl or optionally substituted heterocyclic ring; or alternatively two geminal R⁷ groups can be taken together with the carbon atom to which they are attached to form a spiro, optionally substituted C₃-C₈ cycloalkyl or optionally substituted heterocyclic ring.
